(19) United States
(12) Patent Application Publication
(10) Pub. No.: US 2023/0270735 A1
(43) Pub. Date: Aug. 31, 2023

(54) TOPICAL FORMULATION

(71) Applicant: INCANTHERA (R&D) LTD, Manchester (GB)

(72) Inventor: Kevin Hammond, Wirral Merseyside (GB)

(73) Assignee: INCANTHERA (R&D) LTD, Manchester (GB)

(21) Appl. No.: 18/018,215

(22) PCT Filed: Jul. 27, 2021
(86) PCT No.: PCT/EP2021/071013

§ 371 Date: Jan. 26, 2023

(30) Foreign Application Priority Data
Jul. 27, 2020 (GB) 2011605.9
May 14, 2021 (GB) 2106934.9

(51) Int. Cl. A61K 31/455; A61K 47/10; A61K 47/12; A61K 47/34; A61K 47/26; A61K 9/00; A61K 9/10; A61P 35/00; A61K 8/67; A61Q 17/04

(52) U.S. Cl. CPC A61K 31/455; A61K 47/10; A61K 47/12; A61K 47/34; A61K 47/26; A61K 9/0014; A61K 9/10; A61P 35/00; A61K 8/675; A61Q 17/04; A61K 2800/70

(57) ABSTRACT

The present invention is directed to a topical formulation comprising 1) a dispersed phase comprising: 5 to 15% w/w (percentage weight/weight) nicotinamide; 10 to 60% w/w of a partition coefficient enhancer (Pc enhancer), having a structure of the general formula: CₙH₂ₙ₊₂O₂ where n represents an integer from 3 to 6 inclusive; and ≤10% water and 2) a continuous phase, wherein the formulation has a pH above 4.0. The present invention is also directed to a pharmaceutical composition comprising the topical formulation according to the invention, a pharmaceutical composition according to the invention for use in therapy, and the use of the topical formulation as a cosmetic agent.

### FIELD OF THE INVENTION

The present invention relates to a topical formulation comprising nicotinamide, a pharmaceutical composition comprising the topical formulation according to the invention, a pharmaceutical composition according to the invention for use in therapy, and the use of the topical formulation as a cosmetic agent.

### BACKGROUND OF THE INVENTION

Skin cancers are the most common form of cancers in the developed world, and deaths arising from invasive melanoma are on the increase. UV light has an adverse effect on skin, acting to deactivate the skin's normal repair and protection mechanisms. Evidence suggests that effectively dosed into skin, nicotinamide could protect against this effect by reactivating the skin's own natural repair mechanisms.

Orally administered nicotinamide has been shown in Phase 3 Clinical Studies to have significant preventative effects against actinic (solar) keratosis and there has been subsequent evidence that oral nicotinamide may prevent other more life-threatening skin cancers such as melanoma. The dosing of nicotinamide into skin when administered via an oral route can be calculated from known plasma levels using established procedures. The target level of dosing of nicotinamide—in terms of required flux—required from topical dosing can therefore be established.

However, oral dosing for targeting localised skin disease suffers a number of disadvantages including side effects, such as gastric upset, relatively high amounts of active agent required due to first-pass metabolism, and limitations on bioavailability.

The provision of a topical composition comprising nicotinamide to replace orally administered nicotinamide would be highly beneficial. Academic studies have previously shown relatively high flux levels of nicotinamide into skin is achievable using topical formulations with very high doses or co-solvent systems. But such studies were characterised by compositions wholly unsuitable for practical human use or commercial application. A fully formulated topical product with good aesthetic properties that can achieve the clinically relevant nicotinamide flux levels—in the context of skin cancer prevention—has hitherto not been achieved in spite of the many years of research into topical nicotinamide products. It is well established that for any topical product intended for therapeutic use, compliance is critical to achieving therapeutic benefits. This is most likely to be achieved if products are pleasant to use and have good user-perceived aesthetic properties.

Current topical products intended to provide protection against sun damage rely primarily on sun protection factor (SPF) inclusion. However, the effectiveness of such product in preventing cancerous skin conditions is viewed as having limitations. Moreover, such products can often be aesthetically unpleasant to use and therefore do not encourage compliance which is critical in achieving protection. There remains an unmet need.

To date topical administration of nicotinamide, whilst desirable, has not been successful. This is because prior to the present invention a commercially available formulation, with appropriate consumer acceptability, and capability to deliver therapeutic concentrations of nicotinamide to the target site in the skin has not been available.

It is therefore desirable to provide a topically administered product that is capable of achieving sufficient serum and/or local tissue levels of nicotinamide comparable to the nicotinamide levels achieved with oral dosing. A highly effective trans-dermal nicotinamide delivery from a topical formulation is desirable. Additionally, there is a need for such topical formulation to have good aesthetic properties typically associated with high-end topical cosmetic products.

### SUMMARY OF THE INVENTION

The present invention is defined in the appended claims.

In accordance with a first aspect, there is provided a topical formulation comprising:
a dispersed phase comprising:
   5 to 15% w/w (percentage weight/weight) nicotinamide;
   10 to 60% w/w of a partition coefficient enhancer (Pc enhancer), having a structure of the general formula: CₙH₂ₙ₊₂O₂ where n represents an integer from 3 to 6 inclusive; and
   ≤10% water
and continuous phase,
wherein the formulation has a pH of 4.0 or above.

In accordance with a second aspect, there is provided a pharmaceutical composition comprising the formulation according to the first aspect.

In accordance with a third aspect, there is provided a pharmaceutical composition according to the second aspect for use in therapy.

In accordance with a fourth aspect, there is provided the use of the formulation according to the first aspect as a cosmetic agent.

The present invention provides the possibility of delivering an effective topical product for skin melanoma prevention in subjects suffering from solar keratosis and in preventing recurrence of melanoma in subjects who have previously been treated for such cancers. More broadly, this formulation is expected to provide benefits relating to abnormal skin pigmentation, skin ageing and general damage associated with sun exposure. Therefore, the present invention is directed to topical formulations that may be used in cosmetic compositions and/or pharmaceutical compositions.

Certain embodiments of the present invention may provide one or more of the following advantages:
desired avoidance of possible side effects, such as gastric upset;
desired circumvention of first-pass metabolism and limitations on bioavailability;
desired ease of compliance;
direct targeting of sun exposed tissues and lesions; and
direct treatment of skin lesions.

The details, examples and preferences provided in relation to any particular one or more of the stated aspects of the present invention apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1: Permeation profiles of NAM 0-24 hours from tested formulations in human skin. Each data point represents the mean±SD, n=6.

FIG. 2: Steady state permeation profiles of NAM 6-12 hours from tested formulations Test 1, 2 and 3 and a commercial control in human skin. Each data point represents the mean±SD, n=6. NOTE that mass units permeated per cm² are in μmol/cm² derived from FIG. 1 by dividing mass in ug by 122.1 the molecular weight of NAM.

FIG. 3: Plot of peak plasma concentration, μmol/ml, of NAM plotted versus oral dose in g/M².

FIG. 4: Permeation profiles of NAM 0-24 hours from tested formulations #2.2, #4.2, #6.2, and commercial products TDF, Olay, Paula's choice and SolarCareB3 in human skin. Each data point represents the mean±SD, n=6.

FIG. 5: Permeation profiles of NAM 0-24 hours from formulations with either propyl glycol or butyl glycol in human skin. Each data point represents the mean±SD, n=6.

FIG. 6: The images show an actinic keratosis lesion on the upper cheek of a subject before treatment (left hand panel) and after treatment (right hand panel) with a formulation of the present invention. Reference markings are identified in both the before and after photographs for comparison purposes.

FIG. 7: The images show a basal cell carcinoma on the chest of a subject before treatment (left hand panel) and after treatment (right hand panel) with a formulation of the present invention. Reference markings are identified in both the before and after photographs for comparison purposes.

### DETAILED DESCRIPTION

The present invention provides an advantageous formulation for topical application addressing the problems as noted above.

The present invention is based on the surprising finding that the claimed topical formulation comprising nicotinamide effectively permeates the skin. In many examples, the topical formulations of the present invention have been shown to have a comparable bioavailability to orally administered formulations. Additionally, the topical formulation of the present invention may have good aesthetic properties providing a pleasant feel of the formulation on the skin.

The presently-disclosed subject matter is illustrated by specific but non-limiting embodiments or examples throughout this description. Each example is provided by way of explanation of the present disclosure and is not a limitation thereon.

All combinations of method or process steps as used herein can be performed in any order, unless otherwise specified or clearly implied to the contrary by the context in which the referenced combination is made.

While the following terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Following long-standing patent law convention, the terms “a”, “an”, and “the” refer to “one or more” when used in this application, including the claims.

As used herein, the term “about,” when referring to a value or to an amount of mass, weight, time, volume, concentration or percentage is meant to encompass variations of in some embodiments ±20%, in some embodiments ±15%, in some embodiments ±10%, in some embodiments ±5%, in some embodiments ±1%, in some embodiments 0.5%, and in some embodiments ±0.1% from the specified amount, as such variations are appropriate to perform the disclosed invention(s).

All lists of items, such as, for example, lists of ingredients, are intended to and should be interpreted as Markush groups. Thus, all lists can be read and interpreted as items “selected from the group consisting of” . . . list of items . . . “and combinations and mixtures thereof.”

As used herein, the term “comprises” or “comprising” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”.

The features of any dependent claim may be readily combined with the features of any of the independent claims or other dependent claims. The features of any embodiment may also be readily combined with the features of any other embodiment, unless explicitly referred to otherwise.

### Two Phases

The formulation of the invention can be considered as comprising “two phases” meaning that to prepare the formulation a hydrophilic aqueous/glycol phase and a predominantly hydrophobic oil phase is mixed. “Two-phase” as used in the context of the present disclosure does not necessarily refer to two distinct phases, and amphipathic/amphiphilic agents present will tend to associate at the interface. The two phases according to the present disclosure might for convenience be described in structural terms interchangeably with the aqueous phase (aqueous/glycol phase) being defined as the “dispersed phase”, and the oil phase defined as the “continuous phase”.

In the two phase system described comprises the dispersed phase in the formulation in an amount of about 20% to about 60% w/w, or about 25% to about 50% w/w, or about 35% to about 40%. In some embodiments the dispersed phase is present in the formulation in an amount of about 20% w/w, or about 30% w/w, or about 40% w/w, or about 50% w/w, or about 60% w/w.

The formulation of the present invention comprises an aqueous phase, which may be referred to herein as a dispersed phase. This phase comprises nicotinamide, a partition coefficient enhancer (Pc enhancer) and water at a pH of 4 or above. This phase may also comprise one or more of a polyhydroxy acid, a mild acid and a co-enhancer.

The formulation of the present invention comprises an oil phase, which may be referred to herein as a continuous phase or as an emollient phase. This phase may be selected from suitable oil or emollient phase components known in the art.

### Residual Phase

The residual phase is the combination of components that remains on the skin following evaporation of the volatile components of the oil phase and some water. The residual phase composition, particularly the aqueous/glycol component incorporating the active or therapeutic agent(s), is considered to play an important role in maximising delivery into the skin. Without wished to be bound by theory, it is considered that the high concentration of components and nature of the composition confer favourable thermodynamics such that it becomes energetically favourable for the therapeutic agents to penetrate the skin, facilitated by the penetration enhancer component. The low water content of the formulation favours rapid formation of a favourable residual phase, and the whole formulation is effectively pre-engineered towards effective residual phase formation.

### Nicotinamide

The formulation of the present invention comprises nicotinamide as active agent.

Nicotinamide is soluble in water and may be present in the aqueous phase of the formulation. In an embodiment the aqueous phase is called a non-volatile phase.

Nicotinamide, also known as niacinamide, is a form of vitamin B₃ with IUPAC name pyridine-3-carboxamide. Nicotinamide may be abbreviated as NAM (or N) herein.

### Physicochemical Properties of NAM

| | |
|---|---|
| Chemical structure | 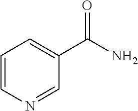 |
| Molecular weight | 122.1 Da |
| Solubility | 1 in 1 of water |
| | ~1 in 5 in propylene glycol 22° C. |
| | ~1 in 3 in propylene glycol 32° C. |
| Log P (_{octanol/water}) | −0.4 |
| Melting point | 128° C. |

The formulation of the present invention comprises 5 to 15% w/w (percentage weight/weight) of nicotinamide. The formulation of the present invention may comprise about 5 to about 12% w/w nicotinamide. Optionally, the formulation of the present invention can comprise about 5.5 to about 11% w/w, and optionally 6 to 10% w/w nicotinamide, or 6 to 12% w/w nicotinamide, 8 to 10% w/w nicotinamide. The formulation can comprise about 5, about 6, about 7, about 8, about 9, about 10, about 11, or about 12% w/w of nicotinamide and preferably about 6, about 8 or about 10% w/w nicotinamide. Formulations of the present invention may comprise more than 5% w/w nicotinamide. Formulations of the present invention may comprise more than 6% w/w, 7% w/w, 8% w/w, 9% w/w, or 10% w/w nicotinamide. Formulations of the present invention may comprise less than 15% w/w nicotinamide. Formulations of the present invention may comprise less than 14% w/w, 13% w/w, 12% w/w, 11% w/w or 10% w/w nicotinamide.

Due to the water solubility of nicotinamide, and the desire for low water content, obtaining topical formulations that are pleasant to use has proven to be a challenge. Typically, high oil containing formulations can be perceived as sticky or greasy and aesthetically unpleasant to use. The stability of nicotinamide has also been challenging. The present inventors have found that by controlling the pH to above 4, a stable nicotinamide formulation can be obtained. The present inventors have also found that a two-phase formulation as described allows topical formulations to be prepared that are pleasant to use.

### Partition Coefficient Enhancer (Pc Enhancer)

Partition coefficient enhancers often described as “penetration enhancers” are used to increase the partitioning of the active into the stratum corneum, thus to increase skin penetration. Increase in penetration is a function of dose of the Pc enhancer due to uptake and clearance from the skin barrier.

Formulations of the present invention comprise about 10 to about 60% w/w of a partition coefficient enhancer (Pc enhancer), having a structure of the general glycol formula: CₙH₂ₙ₊₂O₂ where n represents an integer from 3 to 6 inclusive. The formulation can comprise one or more Pc enhancers. The Pc enhancer may be present in the aqueous phase. The formulation can comprise 15-50, 20-45, 20-50, 25-45, or 30-40% w/w of Pc enhancer. Exemplary Pc enhancers for the formulation of the present invention include propylene glycol, butylene glycol, pentylene glycol, hexylene glycol and 1,5 pentane diol or a combination thereof. In embodiments the formulations comprise propylene glycol or butylene glycol or a combination of propylene glycol and butylene glycol. In a preferred embodiment, the Pc enhancer is propylene glycol.

The Pc enhancers used in the present invention have similar structures to propylene glycol which has been exemplified below and have a general formula: CₙH₂ₙ₊₂O₂ where n represents an integer from 3 to 6 inclusive. Propylene glycol (n=3), butylene glycol (n=4), pentylene glycol (n=5, 1,2 pentane diol), 1,5 pentane diol (n=5) and hexylene glycol (n=6) are in the homologous series CₙH₂ₙ₊₂O₂. Such highly similar structures would be expected to function in a similar way.

Polyhydroxy Acid and/or a Mild Acid

Polyhydroxy acids, as typified by lactobionic acid and gluconolactone, are very effective skin moisturisers and antiaging compounds {Algiert-Zielinska et al., 2019, #18915; Grimes et al., 2004, #61971} and achieve these effects without significant skin irritation {Tasid-Kostov et al., 2019, #60827}. The fundamental mechanism is one of acidification deep into the stratum corneum, for example to inhibit protease activity. Dysfunctional stratum corneum protease activity leads to inhibition of essential stratum corneum lipids and also to reduced stratum corneum integrity/cohesion due to effects on corneodesmosome linking between cells. These effects are entirely complementary with those of nicotinamide.

The formulation of the present invention can comprise one or more polyhydroxy acids. If present, the one or more polyhydroxy acids can provide a further active agent in the formulation. The polyhydroxy acid may be present in the aqueous phase. The formulation can comprise 2 to 10% w/w of a polyhydroxy acid. The formulation can comprise 2 to 9, 2 to 7, 2 to 5, 3 to 5 or about 4% w/w of a polyhydroxy acid. Polyhydroxy acids that may be used in the formulation of the present invention include gluconolactone, galactonolactone, glucuronolactone, galacturonolactone, gulonolactone, ribonolactone, saccharic acid lactone, pantoyllactone, glucoheptonolactone, mannonolactone, and galactoheptonolactone; 2-ketoacids present as free acid. The polyhydroxy acid in the formulation may be selected from gluconolactone, galactose and lactobionic acid. In some embodiments the polyhydroxy acid in the formulation is lactobionic acid. In some embodiments the formulation according to the invention is free from lactobionic acid.

The formulation of the present invention can also comprise one or more mild acids, for purpose of buffering or as additional therapeutic agents. The mild acids according to the present invention are typically acids that partially dissociate. The mild acid may be present in the aqueous phase. The formulation can comprise 2 to 10% w/w of mild acid. The formulation can comprise 2 to 9, 2 to 7, 2 to 5, 3 to 5 or about 4% w/w of mild acid. Mild acids that may be used in the formulation of the present invention include natural fruit acids and alpha hydroxy acids. The mild acid in the formulation may be selected from ascorbic acid, citric acid, glycolic acid, lactic acid, malic acid, tartaric acid and combinations thereof. Polyhydroxy acids may also be considered mild acids as part of the present disclosure.

### Co-Enhancers

Co-enhancers can be used to increase the diffusivity of the active through the stratum corneum barrier layer, thus to increase skin penetration. A co-enhancer may also be referred to as a diffusion coefficient enhancer (Dc enhancer). Much lower doses of the diffusion co-enhancer are required, (but at saturation) believed due to slow clearance from the skin barrier.

In embodiments, the formulation of the present invention may further comprise a co-enhancer. If present, the co-enhancer being amphiphilic in nature will exhibit partial solubility in both the aqueous and oil phases, and the balance of solubility in the two phases is important and characterised by the agents selected herein. The formulation of the present invention can comprise about 0.5 to about 10% w/w of a co-enhancer, selected from the group consisting of a C₁₂ to C₁₄ straight chain fatty acid and a C₁₄ straight chain primary alcohol. The formulation can comprise one or more co-enhancers. Formulations can comprise 0.5 to 7, 0.5 to 6, 0.5 to 5, 1 to 5, 1 to 4, 2 to 4% w/w of Pc enhancer. Exemplary co-enhancers for the formulations of the present invention include myristyl alcohol (1-tetradecanol), lauric acid (dodecanoic acid) and myristic acid (1-tetradecanoic acid).

The co-enhancers used in the formulation of the present invention are generally C₁₂ to C₁₄ straight chain fatty acids and C₁₄ straight chain primary alcohols—all have a very similar structure to 1-tetradecanol which has been exemplified below and thus all have a low solubility in propylene glycol (and the other Pc enhancers used herein) similar to that of 1-tetradecanol. Therefore, they should also be expected to function in a similar way. In more detail, C₆ to C₁₈ straight chain fatty acids and C₆ to C₁₈ straight chain primary alcohols are known to be associated with co-enhancer activity. In the present invention, C₁₂ to C₁₄ straight chain fatty acids and C₁₄ straight chain primary alcohols are used due to their solubility in Pc enhancers of the formula CₙH₍₂ₙ₊₂₎O₂. The solubility of the co-enhancers allows them to achieve the required concentration in solution and to be at or near saturation. This requirement for the co-enhancer to be at or near saturation is the primary determinant of its efficiency as a co-enhancer.

In some embodiments the formulation according to the present invention is substantially free from co-enhancers. In some embodiments the formulation according to the present invention is substantially free from myristyl alcohol. In these embodiments “substantially free from” is understood to mean that the co-enhancers are present in an amount of less than 0.4% w/w, or less than 0.3 w/w, or less than 0.2 w/w, or less than 0.1 w/w, or less than 0.5% w/w, or less than 0.25% w/w in the formulation according to the invention.

### Dispersed Phase

The formulation of the present invention can comprise water. In some embodiments amount of water in the dispersed phase is about 10% w/w or less. In some examples the amount of water in the dispersed phase is about ≤10%, ≤9%, ≤8%, ≤7%, ≤6%, or ≤5% w/w. Water is generally present in the dispersed phase in an amount of about 5% to about 10% w/w. In some embodiments the amount of water in the final formulation is about ≤6%, ≤5%, ≤4%, ≤3%, ≤2%, ≤1%, ≤0.5% w/w. Water is generally present in the final formulation in an amount of about 1% to about 5% w/w

The low water content formulations of the present invention are considered to act to deliver an effective residual phase with favourable thermodynamics for therapeutic agent delivery. The potential drawbacks of high oil content are overcome in the present invention by careful design of the oil phase composition to generate a cream with pleasant in-use and after use properties and encourage compliance.

In the context of this disclosure, the phase comprising water is referred to as the “aqueous phase”, “aqueous/glycol phase” or “dispersed phase”.

### Continuous Phase

The formulation of the present invention comprises a continuous phase. This phase may be selected from suitable oil or emollient phase components known in the art. The continuous phase may be referred to as the “hydrophobic phase” or the “oil phase”.

In embodiments, the continuous phase comprises predominantly hydrophobic components. In some embodiments hydrophobic components may be selected from one or more of silicone elastomers, mineral oils and plant oils.

In embodiments the oil phase may be a phase comprising: 5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and 5 to 45% w/w of a second dimethicone macromer mixture including a methyl siloxane compound and a cross-linked dimethicone macromer.

The first dimethicone macromer mixture can include a polyglycol dimethicone macromer. The first dimethicone macromer mixture can include a compound of the following structure:

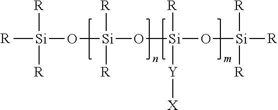

where R represents H or hydrocarbyl group, in particular Cl to C6 alkyl; Y represents a hydrocarbyl group in particular Cl to C6 alkyl group; X represents an amine, a quaternary amino group or acid functionality; and M and n independently represent an integer from 1 to 50.

The first dimethicone macromer mixture can include a dimethicone macromer having a number average molecular weight of more than 1000 (typically more than 2000) and a hydrocarbyl methyl siloxane (generally an alkyl methyl siloxane) having a number average molecular weight of less than 500.

The first dimethicone macromer mixture can include a polyglycol dimethicone macromer cross-linked with a polyalkylene oxide compound (generally a polyethylene glycol compound, a polypropylene glycol compound or a copolymer of ethylene oxide and propylene oxide) or cross-linked with a diene.

The first dimethicone macromer mixture can include a polyglycol dimethicone macromer selected from the group consisting of PEG dimethicone PPG crosspolymer, preferably PEG-12 dimethicone/PPG-20 crosspolymer, and PEG dimethicone bis-isoalkyl PPG crosspolymer.

The first dimethicone macromer mixture can include a polyglycol dimethicone macromer comprising one or more pendant groups from the dimethicone backbone, said pendant group(s) being a polyalkylene oxide group (generally a polyethylene glycol compound, a polypropylene glycol compound or a copolymer of ethylene oxide and propylene oxide).

Optionally the polyglycol dimethicone macromer includes a polyethylene glycol pendant group and a polypropylene glycol pendant group from the dimethicone backbone.

In an embodiment the formulation comprises pyrrolidone carboxylic acid functionalized dimethicone macromer in the first dimethicone macromer mixture.

The formulation can comprise 5 to 45% w/w of the first dimethicone macromer mixture, typically 10 to 40% w/w, generally 20 to 30% w/w.

The first dimethicone macromer mixture comprises 5 to 30% w/w polyglycol dimethicone macromer, typically 10-20% w/w generally 12-19% w/w.

The second dimethicone macromer mixture can include a methyl siloxane compound having a number average molecular weight of less than 1000 and a cross-linked polyalkylsiloxane diol dimethicone macromer having a number average molecular weight of more than 1000, generally of more than 2000.

The formulation can comprise 5 to 45% w/w of the second dimethicone macromer mixture, typically 10 to 40% w/w, generally 20 to 30% w/w.

The second dimethicone macromer mixture can comprise 5 to 30% w/w cross-linked dimethicone macromer, typically 10-20% w/w generally 12-19% w/w.

Further features of the oil phase, the first dimethicone macromer mixture, the second dimethicone macromer mixture and first and second dimethicone macromer mixtures are described in WO 2016/139471 which is incorporated herein by reference.

pH

The formulation of the present invention has a pH of 4 or above. The pH of the formulation of the present invention may be in the range from about 4.0 to about 8.0, or from about 4.5 to about 7.5, or from about 5.0 to about 7.0, or from about 5.0 to about 6.0, or from about 5.0 to about 5.5. In embodiments the formulation has a pH in range of from about 4.0 to about 6.0. The formulation may have a pH greater than about 4.0, 4.5 or 5.0. The formulation may have a pH less than about 6.0, 5.5 or 5.0. The formulation according to the invention may be measured using a standard pH probe, such as a P-10 Sentek pH electrode or a Sentek P-11 electrode.

Controlling the pH between to above 4.0 was found by the inventors to contribute to the stability of the formulation of the invention. For example, it was observed that a formulation with pH=3.8 displayed a significantly inferior stability profile for nicotinamide compared with a formulation with pH=4.5.

### Further Features of the Formulation

In an embodiment the formulation of the present invention does not comprise one or more of the following components: tranexamic acid, zinc salt, zinc salt of a conjugate base of polyhydroxy acid, vitamin D, derivatives of vitamin D, and metabolites of vitamin D, such as calcipotirol.

In embodiments the formulation of the present invention does not comprise tranexamic acid.

In embodiments the formulation of the present invention does not comprise a zinc salt, optionally the formulation does not comprise a zinc salt of a conjugate base of the polyhydroxy acid.

In embodiments the formulation of the present invention does not comprise a cannabidiol.

In embodiments the formulation of the present invention does not comprise vitamin D, derivatives of vitamin D, or metabolites of vitamin D. In some embodiments the formulation of the present invention does not comprise calcipotriol.

In embodiments the formulation comprises a sun-blocking agent. In some embodiments, the sun-blocking agents may also be referred to as sunscreen agents, which may add to the SPF rating. In some embodiments the sun-blocking agent may be a physical agent, a chemical agent or a mixture thereof.

In an embodiment, the present invention provides a topical formulation comprising: a dispersed phase comprising:
5 to 12% w/w (percentage weight/weight) nicotinamide,
2 to 10% w/w of a polyhydroxy acid,
10 to 60% w/w of a partition coefficient enhancer (Pc enhancer), having a structure of the general formula: CnH2n+2O2 where n represents an integer from 3 to 6 inclusive,
about 0.5 to about 10% w/w of a diffusion coefficient enhancer (Dc enhancer), selected from the group consisting of a C12 to C14 straight chain fatty acid and a C14 straight chain primary alcohol, and
≤10% water
   and a continuous phase comprising:
5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
5 to 45% w/w of a second dimethicone macromer mixture including a methyl siloxane compound and a cross-linked dimethicone macromer.

In an embodiment, the present invention provides a topical formulation comprising:

a dispersed phase comprising:
5 to 15% w/w (percentage weight/weight) nicotinamide,
2 to 10% w/w of a polyhydroxy acid and/or mild acid,
10 to 60% w/w of the partition coefficient enhancer (Pc enhancer) propylene glycol
and
≤10% water
   and a continuous phase, wherein the formulation has a pH in the range of 4.0 to 6.0.

In an embodiment, the present invention provides a topical formulation comprising:

a dispersed phase comprising:
5 to 15% w/w (percentage weight/weight) nicotinamide,
2 to 10% w/w of a polyhydroxy acid
10 to 60% w/w of the partition coefficient enhancer (Pc enhancer) propylene glycol
10 to 30% w/w of a particulate sunscreen/sunblock
1 to 5% w/w of skin integrity enhancer linoleic acid
and
≤10% water
   and a continuous phase comprising:
30 to 70% of a blend of caprylyl methicone, PEG-12 dimethicone/PPG-20 crosspolymer, cyclopentasiloxane, dimethicone crosspolymer and trisiloxane
and
wherein the formulation has a pH in the range of 5.0 to 7.0.

In an embodiment, the present invention provides a topical formulation comprising:

a dispersed phase comprising:
5 to 15% w/w (percentage weight/weight) nicotinamide,
2 to 10% w/w of a polyhydroxy acid
2 to 10% w/w of an alpha hydroxy acid lactic acid
10 to 60% w/w of the partition coefficient enhancer (Pc enhancer) propylene glycol
and
≤10% water
   and a continuous phase also comprising:
10 to 30% w/w of sunscreen
and
wherein the formulation has a pH above 4.0.

In an embodiment, the present invention provides a topical formulation comprising:

a dispersed phase comprising:
5 to 15% w/w (percentage weight/weight) nicotinamide,
2 to 10% w/w of a polyhydroxy acid
10 to 60% w/w of the partition coefficient enhancer (Pc enhancer) propylene glycol
1 to 5% w/w of a palmitoyl pentapeptide anti-aging agent
1 to 5% w/w n-acetyl glucosamine skin integrity enhancer
5 to 20% w/w of a particulate sunscreen/sunblock
and
≤10% water
   and a continuous phase
   and
   wherein the formulation has a pH above 4.0.

In an embodiment, the present invention provides a topical formulation comprising:

a dispersed phase comprising:
5 to 15% w/w (percentage weight/weight) nicotinamide,
2 to 10% w/w of a polyhydroxy acid
10 to 60% w/w of the partition coefficient enhancer (Pc enhancer) propylene glycol
1 to 5% w/w of a palmitoyl pentapeptide anti-aging agent
and
≤10% water
   and a continuous phase also incorporating:
5 to 50% w/w of a blend of neopentyl glycol diheptanoate, isodecane, diheptyl
succinate, and a capryloyl glycerin/serbacic acid copolymer
   and
   wherein the formulation has a pH above 4.0.

In an embodiment, the present invention provides a topical formulation comprising:

a dispersed phase comprising:
5 to 15% w/w (percentage weight/weight) nicotinamide,
10 to 60% w/w of the partition coefficient enhancer (Pc enhancer) propylene glycol
10 to 30% w/w of a particulate sunscreen/sunblock
1 to 5% w/w of skin integrity enhancer linoleic acid
1 to 5% w/w of a palmitoyl pentapeptide anti-aging agent
and
≤10% water
   and a continuous phase comprising:
30 to 70% of a blend of caprylyl methicone, PEG-12 dimethicone/PPG-20 crosspolymer, cyclopentasiloxane, dimethicone crosspolymer and trisiloxane
and
wherein the formulation has a pH in the range of 5.0 to 7.0.

### Rationale for an Enhancer Embodiment of the Formulation of the Present Invention

The present invention is, in part, based on the first provision of nicotinamide in a enhancer formulation for topical application to skin. The formulation comprises nicotinamide, a penetration or partition coefficient enhancer (Pc enhancer), having a structure of the general formula: CₙH₂ₙ₊₂O₂ where n represents an integer from 3 to 6 inclusive. A co-enhancer, selected from the group consisting of a C₁₂ to C₁₄ straight chain fatty acid and a C₁₄ straight chain primary alcohol may also be included in the topical formulation. Generally, the formulation comprises 5 to 12% w/w (percentage weight/weight) nicotinamide. Generally, the formulation comprises 10 to 60% w/w of a Pc enhancer. Generally, the formulation comprises, if present, about 0.5 to about 10% w/w of a co-enhancer.

The formulation comprises water. Generally the formulation has about 10% w/w or less of water. Generally, the aqueous phase has about 10% w/w or less of water.

The formulation may also comprise a polyhydroxy acid. Generally, the formulation has 2 to 10% w/w of a polyhydroxy acid, if present.

It is envisioned that the formulation does not comprise tranexamic acid. It is also envisioned that the formulation does not comprise a zinc salt, for example the formulation does not comprise a zinc salt of the conjugate base of the polyhydroxy acid. It is envisioned that the formulation does not comprise vitamin D, derivatives of vitamin D, metabolites of vitamin D, such as calcipotriol.

The present invention is based on the surprising and beneficial properties of the present formulation, which allows the topical formulations to be applied to the skin with a view to preventing and/or treating skin cancers or for use as a cosmetic agent.

The present formulation of nicotinamide in an enhancer system has been carefully designed to achieve flux of nicotinamide into skin from a topical formulation to match the nicotinamide delivery levels previously achieved via oral delivery of nicotinamide in experiments showing prevention and/or treatment of skin cancers. Demonstration of achieving the required level of flux of nicotinamide into human skin with the present formulation is described below. The present formulation of nicotinamide has been designed to achieve this delivery level of nicotinamide with clinically relevant doses of nicotinamide in the formulation: namely 5 to 15% w/w, or 5 to 12% w/w, or 6 to 10% w/w of nicotinamide. The present formulation of nicotinamide has been designed to achieve this delivery level of nicotinamide in the context of a formulation that is aesthetically acceptable and has nice feel in use so that it is acceptable for long term use and patient compliance is more readily achieved.

It should be noted that experimental formulations previously reported, which allegedly achieve flux of nicotinamide into the skin at similar levels to that required herein following topical application, either contain much higher levels of nicotinamide and so are not clinically relevant doses, or contain solvents which are unacceptable for long term use on human skin and hence cannot be marketable products. Additionally, as described below, the present formulation has been demonstrated to show surprisingly good clinical effects.

As nicotinamide is a water-soluble component, achieving a desired low water topical formulation with the benefits discussed in the present invention is known in the art to pose a challenge. The present inventors have found that a combination of nicotinamide in a small amount of water in a two-phase topical system as described leads to the combination of excellent delivery properties and in addition good aesthetic properties of the formulation. Without wishing to be bound by theory, it is thought that the composition of the two-phase system encourages compliance from the subject/patient to use the formulation, through the favourable aesthetics of the continuous phase. Simultaneously, the relatively concentrated aqueous phase comprising a Pc enhancer, such as propylene glycol, and the active agent nicotinamide through its engineered composition gives rise to an effective “residual phase” with favourable thermodynamic properties for enhanced delivery. Critically, also establishing the pH range over which the nicotinamide remains stable in such a formulation, and the whole composition remains stable with respect to consistency and appearance, is seen as a key element. This combination of properties generates a formulation with good stability, favourable in-use and after use properties encouraging compliance, and highly effective delivery of nicotinamide into the skin on application. This efficiency of transfer into the skin is seen in particular when using propylene glycol as the Pc enhancer.

### Enhancer Topical Formulation

Enhancer topical dermatological drug delivery technology may be defined as use of a combination of partition/penetration and diffusion coefficient enhancers, based on Fick's First Law of diffusion. For optimal effectiveness, both the active drug and the diffusion coefficient enhancer should be at, or near, saturated solubility in a non-volatile, thus residual, phase. Co-enhancer gels, formulated as a single-phase solution system comprising a non-volatile co-enhancer residual phase, itself a single phase, volatile solvents and gelled with a suitable cellulose or acrylate polymers are well known, for example; as described in WO 2011/070318 A2 to Reckitt Benckiser Healthcare and U.S. Pat. No. 8,541,470 [symbol] to Futura Medical Developments.

Despite their significant drug delivery benefits, co-enhancer formulations such as those described in WO 2011/070318 A2 and U.S. Pat. No. 8,541,470 are associated with poor rub-in and skin feel, being associated with tackyness and skin drying. Adherence to such topical dermatological medicinal products is disappointingly low. The American dermatologist Dr Steven Feldman concluded in the February 2018 edition of Practical Dermatology that adherence to topical dermatological, in general, is “miserable”.

The UK patent application GB 2549418 “Topical formulations comprising dimethicone macromers” describes a topical formulation technology which aims to address all formulation design-related adherence factors, thus to improve adherence to topical dermatological treatments.

Because formulations of the present invention were required for aesthetic reasons to be creams, rather than gels, thus by definition to contain an oil component, and also because it was required that the oil would not over solubilise the diffusion coefficient enhancer, thus to inhibit enhancement effects, the oil class chosen was of silicone-hybrid chemistry, as described in GB 2549418. Because the chemistry of this class, and silicone fluids in general, are such that they are not miscible with typical glycol partition coefficient enhancers (for example propylene glycol typically at 20-40% w/w of the total formulation) these design requirements force a two-phase design.

### Aqueous Phase of the Cream

In the present formulation the aqueous dispersed phase comprises miscible functional partition (a glycol, such as propylene glycol)—and optionally diffusion coefficient—enhancers. Other miscible non-volatile cosolvents may be added to ensure that both the active drug and the diffusion coefficient enhancer are at, or near, saturated solubility. Water, as a “volatile” solvent or nonsolvent may be added at up to 10% w/w. Although volatile, it will partition into the glycol-dominated “non-volatile” phase, because of its polarity.

### Oil Phase of the Cream

The silicone-based continuous phase comprises a blend typically of three silicone fluids of different volatilities; broadly rapid, intermediate and sustained. Typical silicone fluids are octamethyltrisiloxane, cyclopentasiloxane and the hybrid emollient caprylyl methicone with half-lives of evaporation broadly in the range 10-60 seconds, 15-30 minutes and 1-2 hours, respectively. Caprylyl methicone, may be part of the continuous phase and with an evaporation rate consistent with twice-a-day application. The diffusion coefficient enhancer, typically a C12-14 acid or C14 alcohol will, because of this chemistry, partition between the glycol and silicone phases and will be, ideally, saturated in both.

### Structuring of the Cream

The cream can be structurally defined as an aqueous/glycol-in-silicone/oil dispersion; thus with droplets of the aqueous/glycol phase dispersed in the hydrophobic oil/silicone fluids by use of, typically, PEG-12 Dimethicone/PPG-20 type Crosspolymer. Typically a dimethicone crosspolymer will also be added to modify the viscosity (lightness of touch) of the final cream. On application to the skin the consumer experiences the soft and silky skin feel of the mixed silicone elastomer-silicone fluid/oil continuous phase. Loss of volatile silicones occurs quickly to give the perception of absorption into the skin.

### General Process for Manufacturing the Formulation

In summary the manufacture of a formulation of the present invention involves preparation of a first pre-mix which comprises the nicotinamide, the Pc enhancer and water, optionally the polyhydroxy acid, and finally, the co-enhancer, if present. The first pre-mix represents the aqueous, dispersed phase of the formulation. The components are mixed together and may also be gently heated (>35° C.) if the polyhydroxy acid and the co-enhancer are present, to ensure the polyhydroxy acid and co-enhancer are dissolved. A second pre-mix comprises components of the oil continuous phase and is added to further components of the oil phase and mixed together. The first pre-mix, comprising components of the aqueous/glycol phase, is added slowly and with vigorous mixing to the second pre-mix, comprising components of the continuous phase until a firm cream has been formed.

### Treatment Using the Formulation

The present invention provides a method of preventing and/or treating a skin cancers, the method comprising topical application to skin of the formulation of the present invention.

The present invention provides the formulation of the present invention for use in therapy. The present invention also provides the formulation of the present invention for use in a method of preventing and/or treating a skin cancer. The present invention provides the formulation of the present invention for topical application for use in a method of preventing and/or treating a skin cancer.

The present invention also provides use of the formulation of the present invention in the manufacture of a medicament for the prevention and/or treatment of a skin cancer.

The present invention also provides a pharmaceutical composition comprising the formulation of the present invention to prevent or treat a skin cancer.

The skin cancer to be prevented or treated can be selected from actinic (solar) keratosis, basal cell carcinoma, squamous cell carcinoma and melanoma.

Therefore present invention i) provides a method of, ii) provides a formulation for use in a method of, iii) provides use of a formulation in the manufacture of a medicament and/or iv) a pharmaceutical composition comprising a formulation, for preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.

A benefit of the formulation is that it may only need to be applied to skin once or twice a day and hence the method may comprise, consist essentially of or consist of application to skin once or twice a day.

The formulation is for topical application to skin. It is envisaged that the skin is human skin.

In certain embodiments, the topical formulation and/or pharmaceutical composition may have one or more of the following effects:
stable topical formulation comprising nicotinamide;
good transfer of nicotinamide into the skin;
aesthetically pleasing formulation;
formulation that is pleasant to use;
protection from the sun;
efficient delivery of nicotinamide to the site to be treated;
effective treatment of skin cancer; and
effective treatment of skin lesions.

The present disclosure may be described by one or more of the following paragraphs:
A. A formulation for topical application and for use in a method of preventing and/or treating a skin cancer,
the formulation comprising:
a non-volatile residual phase comprising:
5 to 12% w/w (percentage weight/weight) nicotinamide,
   2 to 10% w/w of a polyhydroxy acid,
10 to 60% w/w of a partition coefficient enhancer (Pc enhancer), having a structure of the general formula: CₙH₂ₙ₊₂O₂ where n represents an integer from 3 to 6 inclusive,
   about 0.5 to about 10% w/w of a diffusion coefficient enhancer (Dc enhancer), selected from the group consisting of a C₁₂ to C₁₄ straight chain fatty acid and a C₁₄ straight chain primary alcohol, and
≤10% water
and an emollient phase comprising:
5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
   5 to 45% w/w of a second dimethicone macromer mixture including a methyl siloxane compound and a cross-linked dimethicone macromer.
B. The formulation for the use according to paragraph A, wherein the formulation comprises 5.5 to 11% w/w, and optionally 6 to 10% w/w nicotinamide.
C. The formulation for the use according to paragraph A or B, wherein the Pc enhancer is selected from the group consisting of propylene glycol, butylene glycol, pentylene glycol, hexylene glycol, and 1,5 pentane diol and a combination thereof.
D. The formulation for the use according to any one of paragraphs A to C, wherein the formulation comprises 15-50, 20-45, 25-45, or 30-40% w/w of the Pc enhancer.
E. The formulation for the use according to any one of the preceding paragraphs, wherein the Dc enhancer is selected from the group consisting of myristyl alcohol (1-Tetradecanol), myristic acid, and lauric acid (dodecanoic acid).
F. The formulation for the use according to any one of the preceding paragraphs, wherein the Dc enhancer is 0.5-5, 1-5, 2-4% w/w (percentage weight/weight) of the formulation.
G. The formulation for the use according to any one of the preceding paragraphs, wherein the formulation comprises 2 to 9, 2 to 7, 2 to 5, 3 to 5 or about 4% w/w of a polyhydroxy acid.
H. The formulation for the use according to any one of the preceding paragraphs, wherein the polyhydroxy acid is selected from gluconolactone, galactose and lactobionic acid.
I. The formulation for the use according to any of the preceding paragraphs wherein the first dimethicone macromer mixture includes a polyglycol dimethicone macromer.
J. The formulation for the use according to any of the preceding paragraphs wherein the first dimethicone macromer mixture includes a compound of the following structure:

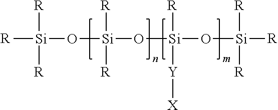

Where R represents H or hydrocarbyl group, in particular Cl to C6 alkyl
   Y represents a hydrocarbyl group in particular Cl to C6 alkyl group;
   X represents an amine, a quaternary amino group or acid functionality.
   M and n independently represent an integer from 1 to 50.
K. The formulation for the use according to any of the preceding paragraphs wherein the first dimethicone macromer mixture includes a dimethicone macromer having a number average molecular weight of more than 1000 (typically more than 2000) and a hydrocarbyl methyl siloxane emollient (generally an alkyl methyl siloxane) having a number average molecular weight of less than 500.
L. The formulation for the use according to any of the preceding paragraphs wherein the first dimethicone macromer mixture includes a polyglycol dimethicone macromer cross-linked with a polyalkylene oxide compound (generally a polyethylene glycol compound, a polypropylene glycol compound or a copolymer of ethylene oxide and propylene oxide) or cross-linked with a diene.
M. The formulation for the use according to paragraph L, wherein the first dimethicone macromer mixture includes a polyglycol dimethicone macromer selected from the group consisting of PEG dimethicone PPG crosspolymer, preferably PEG-12 dimethicone/PPG-20 crosspolymer, and PEG dimethicone bis-isoalkyl PPG crosspolymer.
N. The formulation for the use according to any of the preceding paragraphs wherein the first dimethicone macromer mixture includes a polyglycol dimethicone macromer comprising one or more pendant groups from the dimethicone backbone, said pendant group(s) being a polyalkylene oxide group (generally a polyethylene glycol compound, a polypropylene glycol compound or a copolymer of ethylene oxide and propylene oxide).
O. The formulation for the use according to paragraph N wherein the polyglycol dimethicone macromer includes a polyethylene glycol pendant group and a polypropylene glycol pendant group from the dimethicone backbone.
P. The formulation for the use according to any one of paragraphs J to O comprising pyrrolidone carboxylic acid functionalized dimethicone macromer.
Q. The formulation for the use according to any of the preceding paragraphs comprising 5 to 45% w/w first dimethicone macromer mixture, typically 10 to 40% w/w, generally 20 to 30% w/w.
R. The formulation for the use according to any of the preceding paragraphs wherein the second dimethicone macromer mixture includes a methyl siloxane compound having a number average molecular weight of less than 1000 and a cross-linked polyalkylsiloxane diol dimethicone macromer having a number average molecular weight of more than 1000, generally of more than 2000.
S. The formulation for the use according to any of the preceding paragraphs comprising 5 to 45% w/w second dimethicone macromer mixture, typically 10 to 40% w/w, generally 20 to 30% w/w.
T. The formulation for the use according to any of the preceding paragraphs where the first dimethicone macromer mixture comprises 5 to 30% w/w polyglycol dimethicone macromer, typically 10-20% w/w generally 12-19% w/w.
U. The formulation for the use as embodied in any preceding paragraph where the second dimethicone macromer mixture comprises 5 to 30% w/w cross-linked dimethicone macromer, typically 10-20% w/w generally 12-19% w/w.
V. The formulation for the use according to any of the preceding paragraphs wherein the formulation does not comprise tranexamic acid.
W. The formulation for the use according to any of the preceding paragraphs wherein the formulation does not comprise a zinc salt, optionally the formulation does not comprise a zinc salt of a conjugate base of the polyhydroxy acid.
X. The formulation for the use according to any of the preceding paragraphs wherein the skin cancer is selected from actinic keratosis, basal cell carcinoma, squamous cell carcinoma and melanoma.
Y. The formulation for the use according to any of the preceding paragraphs wherein the method of preventing and/or treating a skin cancer is selected from a method of preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.
Z. The formulation for the use of any one of the preceding paragraphs, wherein the method consists of application to skin once a day or twice a day.
AA. Use of a formulation in the manufacture of a medicament for the prevention and/or treatment of a skin cancer, the formulation comprising:
a. a non-volatile residual phase comprising:
i. 5 to 12% w/w (percentage weight/weight) nicotinamide,
ii. 2 to 10% w/w of a polyhydroxy acid,
iii. 10 to 60% w/w of a partition coefficient enhancer (Pc enhancer), having a structure of the general formula: CₙH₂ₙ₊₂O₂ where n represents an integer from 3 to 6 inclusive,
iv. about 0.5 to about 10% w/w of a diffusion coefficient enhancer (Dc enhancer), selected from the group consisting of a C₁₂ to C₁₄ straight chain fatty acid and a C₁₄ straight chain primary alcohol, and ≤10% water
b. and an emollient phase comprising:
i. 5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
ii. 5 to 45% w/w of a second dimethicone macromer mixture including a methyl siloxane compound and a cross-linked dimethicone macromer.
BB. The use according to paragraph AA, wherein the formulation is as further defined in paragraphs B to W.
CC. The use according to paragraph AA, wherein the skin cancer is selected from actinic keratosis, basal cell carcinoma, squamous cell carcinoma and melanoma.
DD. The use according to paragraph AA, wherein the prevention and/or treatment of a skin cancer is selected from a method of preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.
EE. The use according to any one of paragraphs AA to DD, wherein the treating and/or preventing consists of application to human skin once a day or twice a day.
FF. A method of preventing and/or treating a skin cancer, the method comprising topical application to skin of a formulation comprising:
a. a non-volatile residual phase comprising:
i. 5 to 12% w/w (percentage weight/weight) nicotinamide,
ii. 2 to 10% w/w of a polyhydroxy acid,
iii. 10 to 60% w/w of a partition coefficient enhancer (Pc enhancer), having a structure of the general formula: CₙH₂ₙ₊₂O₂ where n represents an integer from 3 to 6 inclusive,
iv. about 0.5 to about 10% w/w of a diffusion coefficient enhancer (Dc enhancer), selected from the group consisting of a C₁₂ to C₁₄ straight chain fatty acid and a C₁₄ straight chain primary alcohol, and ≤10% water
b. and an emollient phase comprising:
i. 5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
5 to 45% w/w of a second dimethicone macromer mixture including a methyl siloxane compound and a cross-linked dimethicone macromer.
GG. The method according to paragraph FF, wherein the skin cancer is selected from the group consisting of actinic keratosis, basal cell carcinoma, squamous cell carcinoma and melanoma.
HH. The method according to paragraph FF, wherein the preventing and/or treating is selected from the group consisting of a method of preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.
II. The method according to paragraph FF, wherein the method consists of application to skin once or twice a day.
JJ. The method according to paragraph FF, wherein the skin is human skin.
KK. The method according to paragraph FF, wherein the formulation comprises 5.5 to 11% w/w, and optionally 6 to 10% w/w nicotinamide.
LL. The method according to paragraph FF, wherein the Pc enhancer is selected from the group consisting of propylene glycol, butylene glycol, pentylene glycol, hexylene glycol, and 1,5 pentane diol and a combination thereof.
MM. The method according to paragraph FF, wherein the formulation comprises 15-50, 20-45, 25-45, or 30-40% w/w of the Pc enhancer.
NN. The method according to paragraph FF, wherein the Dc enhancer is selected from the group consisting of myristyl alcohol (1-Tetradecanol), myristic acid, and lauric acid (dodecanoic acid).
OO. The method according to paragraph FF, wherein the Dc enhancer is 0.5-5, 1-5, 2-4% w/w (percentage weight/weight) of the formulation.
PP. The method according to paragraph FF, wherein the formulation comprises 2 to 9, 2 to 7, 2 to 5, 3 to 5 or about 4% w/w of a polyhydroxy acid.
QQ. The method according to paragraph FF, wherein the polyhydroxy acid is selected from gluconolactone, galactose and lactobionic acid.
RR. The method according to paragraph FF, wherein the first dimethicone macromer mixture includes a polyglycol dimethicone macromer.
SS. The method according to paragraph FF, wherein the first dimethicone macromer mixture includes a compound of the following structure:

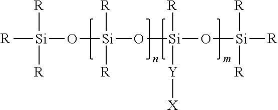

Where R represents H or hydrocarbyl group, in particular Cl to C6 alkyl
   Y represents a hydrocarbyl group in particular Cl to C6 alkyl group;
   X represents an amine, a quaternary amino group or acid functionality.
   M and n independently represent an integer from 1 to 50.
TT. The method according to paragraph FF, wherein the first dimethicone macromer mixture includes a dimethicone macromer having a number average molecular weight of more than 1000 (typically more than 2000) and a hydrocarbyl methyl siloxane emollient (generally an alkyl methyl siloxane) having a number average molecular weight of less than 500.
UU. The method according to paragraph FF, wherein the first dimethicone macromer mixture includes a polyglycol dimethicone macromer cross-linked with a polyalkylene oxide compound (generally a polyethylene glycol compound, a polypropylene glycol compound or a copolymer of ethylene oxide and propylene oxide) or cross-linked with a diene.
VV. The method according to paragraph FF, wherein the first dimethicone macromer mixture includes a polyglycol dimethicone macromer selected from the group consisting of PEG dimethicone PPG crosspolymer, preferably PEG-12 dimethicone/PPG-20 crosspolymer, and PEG dimethicone bis-isoalkyl PPG crosspolymer.
WW. The method according to paragraph FF, wherein the first dimethicone macromer mixture includes a polyglycol dimethicone macromer comprising one or more pendant groups from the dimethicone backbone, said pendant group(s) being a polyalkylene oxide group (generally a polyethylene glycol compound, a polypropylene glycol compound or a copolymer of ethylene oxide and propylene oxide).
XX. The method according to paragraph FF, wherein the polyglycol dimethicone macromer includes a polyethylene glycol pendant group and a polypropylene glycol pendant group from the dimethicone backbone.
YY. The method according to paragraph FF, comprising pyrrolidone carboxylic acid functionalized dimethicone macromer.
ZZ. The method according to paragraph FF, comprising 5 to 45% w/w first dimethicone macromer mixture, typically 10 to 40% w/w, generally 20 to 30% w/w.
AAA. The method according to paragraph FF, wherein the second dimethicone macromer mixture includes a methyl siloxane compound having a number average molecular weight of less than 1000 and a cross-linked polyalkylsiloxane diol dimethicone macromer having a number average molecular weight of more than 1000, generally of more than 2000.
BBB. The method according to paragraph FF comprising 5 to 45% w/w second dimethicone macromer mixture, typically 10 to 40% w/w, generally 20 to 30% w/w.
CCC. The method according to paragraph FF, where the first dimethicone macromer mixture comprises 5 to 30% w/w polyglycol dimethicone macromer, typically 10-20% w/w generally 12-19% w/w.
DDD. The method according to paragraph FF, where the second dimethicone macromer mixture comprises 5 to 30% w/w cross-linked dimethicone macromer, typically 10-20% w/w generally 12-19% w/w.
EEE. The method according to paragraph FF, wherein the formulation does not comprise tranexamic acid.
FFF. The method according to paragraph FF, wherein the formulation does not comprise a zinc salt, optionally the formulation does not comprise a zinc salt of a conjugate base of the polyhydroxy acid.

The present disclosure may also be described by one or more of the following paragraphs:
a. A topical formulation comprising:
   an aqueous phase comprising:
   i. 5 to 15% w/w (percentage weight/weight) nicotinamide,
   ii. 10 to 60% w/w of a partition coefficient enhancer (Pc enhancer), having a structure of the general formula: CₙH₂ₙ₊₂O₂ where n represents an integer from 3 to 6 inclusive,
   iii. and
   iv. ≤10% water
      and an oil phase,
   wherein the formulation has a pH in the range 4.0 to 6.0.
b. The formulation according to paragraph a, wherein the formulation comprises 5 to 12% w/w, 5.5 to 11% w/w, and optionally 6 to 10% w/w nicotinamide.
c. The formulation according to paragraph a or paragraph b, wherein the Pc enhancer is selected from the group consisting of propylene glycol, butylene glycol, pentylene glycol, hexylene glycol, and 1,5 pentane diol and a combination thereof.
d. The formulation according to any one of paragraphs a to c, wherein the formulation comprises 15-50% w/w, 20-45% w/w, 25-45% w/w, or 30-40% w/w of the Pc enhancer.
e. The formulation according to any one of the preceding paragraphs, wherein the formulation further comprises about 0.5 to about 10% w/w of a diffusion coefficient enhancer (Dc enhancer), selected from the group consisting of a C₁₂ to C₁₄ straight chain fatty acid and a C₁₄ straight chain primary alcohol,
   optionally the Dc enhancer is selected from the group consisting of myristyl alcohol (1-Tetradecanol), myristic acid, and lauric acid (dodecanoic acid).
f. The formulation according to paragraph e, wherein the Dc enhancer is 0.5-5, 1-5, 2-4% w/w (percentage weight/weight) of the formulation.
g. The formulation according to any one of the preceding claims, wherein the formulation further comprises 2 to 10% w/w of a polyhydroxy acid,
   optionally the formulation comprises 2 to 9, 2 to 7, 2 to 5, 3 to 5 or about 4% w/w of the polyhydroxy acid.
h. The formulation according to paragraph g, wherein the polyhydroxy acid is selected from gluconolactone, galactose and lactobionic acid.
i. The formulation according to any one of the preceding paragraphs, wherein the oil phase of the formulation is an emollient phase comprising:
   5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
   5 to 45% w/w of a second dimethicone macromer mixture including a methyl siloxane compound and a cross linked dimethicone macromer.
j. The formulation according to any of the preceding paragraphs wherein the formulation does not comprise tranexamic acid.
k. The formulation according to any of the preceding paragraphs wherein the formulation does not comprise a zinc salt, optionally the formulation does not comprise a zinc salt of a conjugate base of the polyhydroxy acid.
l. The formulation according to any of paragraphs a to k for use in therapy.
m. The formulation according to any of paragraphs a to k for use in a method of preventing and/or treating a skin cancer.
n. The formulation for the use of paragraph m wherein the skin cancer is selected from actinic keratosis, basal cell carcinoma, squamous cell carcinoma and melanoma.
o. The formulation for the use of paragraph m wherein the method of preventing and/or treating a skin cancer is selected from a method of preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.
p. The formulation for the use of any one of paragraphs l to o, wherein the method consists essentially of application to skin once a day or twice a day
q. The formulation for the use of any one of paragraphs l to o, wherein the skin is human skin.
r. A method of preventing and/or treating a skin cancer, the method comprising topical application to skin of the formulation of paragraph a.
s. The method according to paragraph r, wherein the skin cancer is selected from the group consisting of actinic keratosis, basal cell carcinoma, squamous cell carcinoma and melanoma.
t. Use of a formulation according to any of paragraphs a to k in the manufacture of a medicament for preventing and/or treating a skin cancer.
u. The use according to paragraph t, wherein the skin cancer is selected from actinic keratosis, basal cell carcinoma, squamous cell carcinoma and melanoma.
v. A pharmaceutical composition comprising the formulation according to paragraphs a to k to prevent and/or treat a skin cancer.
w. The pharmaceutical composition according to paragraph v, wherein the skin cancer is selected from actinic keratosis, basal cell carcinoma, squamous cell carcinoma and melanoma.

### EXAMPLES

### Example of Method for Making Exemplary Formulations of 6-10% Nicotinamide

TABLE 1

| | | % w/w | | % w/w | | % w/w | |
|---|---|---|---|---|---|---|---|
| | | Serum | | Serum | | Serum | |
| | % w/w | #2 | | #4 | % w/w | #6 | |
| | Serum | 500 g | | 500 g | Serum | 500 g | |
| | #2 | actual | | actual | #6 | actual | Process |
| Material | 500 g | Test 1 | 150.0 | Test 2 | 500 g | Test 3 | Order |
| Lactobionic | 22.50 | 22.50 | 30.00 | 30.00 | 37.50 | 37.49 | 7 |
| Acid (LBA) | | | | | | | |
| Gluconolactone | 7.50 | 7.51 | 10.00 | 10.03 | 12.50 | 12.51 | 6 |
| (GLA) | | | | | | | |
| % Polyhydroxy | 6.00 | | 8.00 | | 10.00 | | |
| acid (PHA) | | | | | | | |
| Nicotinamide (N) | 30.00 | 30.00 | 40.00 | 39.99 | 50.00 | 50.02 | 5 |
| % N | 6.00 | | 8.00 | | 10.00 | | |
| NaCl | 2.00 | 2.00 | 2.00 | 2.00 | 2.00 | 2.0 | 1 |
| Glycerol (G) | 17.50 | 17.49 | 2.50 | 2.52 | 0.00 | 0.00 | 3 |
| Propylene | 150.00 | 150.00 | 175.00 | 175.02 | 200.00 | 200.03 | 4 |
| glycol (PG) | | | | | | | |
| % PG | 30.00 | | 35.00 | | 40.00 | | |
| De-ionized | 28.50 | 28.51 | 28.50 | 28.51 | 8.50 | 8.53 | 2 |
| water (DI H2O) | (16.81%) | | | | (5.01%) | | |
| Myristyl alcohol | 7.50 | 7.55 | 7.50 | 7.58 | 7.50 | 7.53 | 8 |
| Phenoxyethanol | 2.50 | 2.51 | 2.50 | 2.50 | 2.50 | 2.50 | 9 |
| Trisiloxane | 50.00 | 50.04 | 45.00 | 45.01 | 35.00 | 35.00 | 11 |
| Carbomer | 2.00 | 2.00 | 2.00 | 2.00 | 2.00 | 2.01 | 12 |
| EL-7040 | 120.00 | 120.24 | 103.00 | 103.23 | 94.50 | 94.58 | 10 |
| ST-Elastomer/9040 | 60.00 | 60.36 | 52.00 | 52.32 | 48.00 | 48.25 | 13 |
| Silicone | | | | | | | |
| Elastomer | | | | | | | |
| Total | 500.00 | 500.71 | 500.00 | 500.71 | 500.00 | 500.45 | |
| pH | | 3.827 | | 3.828 | | 3.931 | |

### Process:

At the 500 g lab scale the batch is made using two premixes
V1 (400 ml): Premix 1
NaCl+DI H20+LEA+GLA+G+PG+Nicotinamide (as a 6 ingredient stock) +Phenoxyethanol+Myristyl alcohol (014 alcohol aka tetradecanol); gentle heat to 35° C. to dissolve; cool to <25° C.
V2 (100 ml): Premix 2
Carbomer+trisiloxane; mix to disperse and also on addition to V3
V3 (Kenwood bowl) Final mix with beater blade
   EL-7040 (Caprylyl Methicone (and) PEG-12 Dimethicone/PPG-20 Crosspolymer
   into Kenwood bowl
   add contents of V2 (Carbomer+trisiloxane) from low speed increasing speed, then speed at #6 max 2 minutes to form “syrup”.
   Add V1 dropwise/slow ribbon over ˜10-12 minutes, speed at #6 (max). Phase is inverted when the serum is peaked and firm when running at #6.
   Clean sides to middle, add ST-E (Cyclopentasiloxane (and) Dimethicone Crosspolymer/9040 Silicone Elastomer, mix speed #6, 3 minutes, clean sides to middle, #6 further 3 minutes.
   Pack into 500 g sealed buckets
Note on Premix 1:
   Order of addition as shown
   After Niacinamide addn, tumble mixed into clear solution
   After GLA addn, tumble mixed into clear solution
   After LBA addn, tumble mixed into clear solution
   Gentle heat to dissolve last traces of LBA if required (10% PHA only)
   Then add Phenoxyethanol and Myristyl alcohol (C14 alcohol, aka tetradecanol) to complete Premix 1

### Further Exemplary Formulations of 6-10% Nicotinamide

TABLE 2
Formulation examples from manufacturing batch sheets (actual batch weights)

| | Cream #2.2 | Cream #4.2 | Cream #6.2 | Cream #7.1 | Cream #9.l |
|---|---|---|---|---|---|
| | g/500 g | g/500 g | g/500 g | g/500 g | g/500 g |
| | actual | actual | actual | actual | actual |
| | DOM: 26- | DOM: 26- | DOM: 26- | DOM: 26- | DOM: 26- |
| Material | 30/04/2020 | 30/04/2020 | 30/04/2020 | 30/04/2020 | 30/04/2020 |
| Lactobionic Acid | 22.50 | 30.01 | 37.50 | 22.51 | 15.00 |
| Gluconolactone | 7.52 | 10.00 | 12.53 | 7.50 | 5.01 |
| % PHA (polyhydroxy | 6.00 | 8.00 | 10.00 | 6.00 | 4.00 |
| acid) | | | | | |
| Nicotinamide (N) | 30.02 | 40.00 | 50.02 | 50.02 | 50.02 |
| % Nicotinamide | 6.00 | 8.00 | 10.00 | 10.00 | 10.00 |
| Sodium chloride solid | 2.00 | 2.00 | 2.02 | 2.00 | 2.02 |
| Glycerol | 17.51 | 2.50 | 0.00 | 0.00 | 0.00 |
| Propylene glycol | 150.02 | 175.11 | 200.01 | 200.02 | — |
| Butylene glycol | — | — | — | — | 200.03 |
| % prop/butylene | 30.00 | 35.00 | 40.00 | 40.00 | 40.00 |
| glycol | | | | | |
| DI H20 | 28.52 | 28.53 | 8.49 | 8.53 | 50.00 |
| Myristyl alcohol | 7.53 | 7.50 | 7.51 | 7.49 | — |
| Lauric acid | — | — | — | — | 7.49 |
| Phenoxyethanol | 2.51 | 2.51 | 2.52 | 2.53 | 2.51 |
| 25% LA NH3 (liquid | 2.00 | 2.01 | 2.00 | 1.00 | 1.20 |
| ammonia) | | | | | |
| (state after LA NH3) | Clear | Clear | Clear | Clear | Clear |
| Octamethyltrisiloxane | 48.03 | 43.07 | 33.02 | 44.01 | 20.01 |
| Carbomer | 2.00 | 2.00 | 1.99 | 2.00 | 2.00 |
| EL-7040Hydro | 120.09 | 103.20 | 94.65 | 101.77 | 96.14 |
| Elastomer blend¹ | | | | | |
| 9040 Elastomer | 60.15 | 52.16 | 48.20 | 50.85 | 48.12 |
| blend² | | | | | |
| Total | 500.40 | 500.60 | 500.46 | 500.23 | 499.55 |
| pH after LA NH3 | 4.471 | 4.423 | 4.610 | 4.592 | 4.460 |
| pH serum (4.4-4.6) | 4.490 Pass | 4.480 Pass | 4.514 Pass | 4.495 Pass | 4.447 Pass |

¹Caprylyl Methicone (and) PEG-12 Dimethicone/PPG-20 Crosspolymer
²Cyclopentasiloxane (and) Dimethicone Crosspolymer

Treatment and Prevention of Skin Cancers and Conditions Associated with UV Damage

Formulations described herein are part of the invention and are for use in therapy. Formulations of the present invention are for use in a method of preventing and/or treating an ultraviolet (UV) radiation-induced skin condition, or for preventing and/or treating a skin cancer. UV damage to skin can include discolouration, and age-related brown spots (abnormal production or deposition of melanin arising from UV exposure) which may lead to cancers. More severe UV damage can include lesion formation and pre-cancerous lesions. Skin cancers can include actinic keratosis, basal cell carcinoma, squamous cell carcinoma and melanoma. Therefore, the formulation of the present invention is for use in a method of treating or preventing actinic keratosis, basal cell carcinoma, squamous cell carcinoma and melanoma. In particular the formulation of the present invention is for use in a method for use in a method of preventing actinic keratosis, treating actinic keratosis, preventing melanoma, preventing recurrence of melanoma, treating melanoma, preventing squamous cell carcinoma, preventing basal cell carcinoma, treating squamous cell carcinoma and treating basal cell carcinoma.

The present invention further includes use of the formulation of the present invention in the manufacture of a medicament for the treatment or prevention of an ultraviolet radiation-induced skin condition, or the treatment or prevention of a skin cancer. Treatment or prevention of a skin cancer can include prevention of actinic keratosis, treatment of actinic keratosis, prevention of melanoma, prevention of the recurrence of melanoma, treatment of melanoma, prevention of squamous cell carcinoma, prevention of basal cell carcinoma, treatment of squamous cell carcinoma or treatment of basal cell carcinoma.

The present invention provides a method of treating or preventing an ultraviolent radiation-induced skin by topical application to skin of a formulation of the present invention. The present invention further provides a method of treating or preventing an ultraviolent radiation-induced skin condition, the method comprising topical application to skin of a formulation of the present invention. The present invention also provides a method of preventing actinic keratosis, treating actinic keratosis, preventing melanoma, preventing recurrence of melanoma, treating melanoma, preventing squamous cell carcinoma, preventing basal cell carcinoma, treating squamous cell carcinoma and treating basal cell carcinoma comprising topical application to skin of a formulation of the present invention.

In embodiments formulations of the present invention need only be applied to skin once or twice a day. Generally the skin is human skin.

The convenience of once-a day application is widely appreciated by users. As described herein, as the dose of the Pc enhancer is increased its effect to increase skin penetration is extended over time, thus providing the ability to achieve once or twice-a-day dosing.

### The Cosmetic and Medicinal Use of Nicotinamide in Dermatology

The use of Nicotinamide (NAM) in cosmetic and medicinal dermatology, via topical or oral administration, extends back nearly 50 years and has been documented in several major reviews (1-6).

Mostly, NAM is used topically and for cosmetic purposes. NAM, both in vitro, and at concentrations as low as 1-10 μmol L⁻¹ (75%-100% of maximum response), and in vivo topically in man, very significantly increases the biosynthesis of ceramides and other stratum corneum lipids to increase skin barrier function (7). Consistent with this, Soma Y et al. found that 2% NAM cosmetic cream was a more effective moisturiser than standard petrolatum in a group of 28 patients with atopic dermatitis (8). Similarly, Draelos Z D et al. (9) reported that a 2% NAM-containing facial moisturizer improved skin barrier in subjects with rosacea in a non-treated controlled study. Also, Kawada A et al. (10) demonstrated facial anti-wrinkle effects of a cosmetic containing 4% NAM compared with a placebo control. Finally, in their review, Wohlrab and Kreft (4) conclude that the antipruritic effects of NAM are mainly based on barrier-protective effects.

The efficacy of NAM in medicinal inflammatory dermatological conditions such as bullous pemphigoid (11), psoriasis (12), pemphigus vulgaris (13) and rosacea (14) may be due in part to its inhibition of the nuclear poly(ADP-ribose) polymerase-1 (PARP-1). Nuclear poly(ADP-ribose) polymerase-1 (PARP-1) NFκB-mediated transrepression is important in reducing the expression of adhesion molecules and pro-inflammatory mediators such as IL-12, TNF-α, IL-1 and nitric oxide.

Ultraviolet radiation-induced skin cancers, including basal cell carcinoma (BCC), squamous cell carcinoma (SCC) and melanoma (MEL) are a global public health issue (15). Oncogenic mechanisms include direct damage to DNA, suppression of the skin's immunity and energy depletion within the skin, all of which are reversed by NAM (16). In 2012, Surjana et al, (17) reported on two placebo-controlled studies in immune-competent volunteers with the pre-cancerous skin condition actinic keratoses (AK). In total, 37 patients each per active NAM or placebo treatment were treated over 4 months with either oral NAM (500 mg twice daily, study 1, or 500 mg once daily, study 2). At the end of 4 months, highly statistically significant reductions in AK versus baseline (35% study 1; 29% study 2) were seen with the Nicotinamide groups compared with placebo. However, Moloney et al. (35) found no difference in AK scores after 6 months topical treatment with 1% Nicotinamide cream or placebo. Given the finding of Surjana et al, (17) with oral high dose oral NAM it seems likely that the low dose applied (and delivery efficiency) from the 1% NAM cream may have contributed to the lack of effect in AK.

In a continuation of the oral NAM program, in 2015, Chen et al. (18) published the results of the ONTRAC study on participants selected on the basis of having had at least two nonmelanoma skin cancers in the last five years. The 386 participants were randomly assigned to receive oral NAM 500 mg twice a day or placebo over a 12 months treatment period. At 12 months assessment, the rate of new nonmelanoma skin cancers was statistically significantly lowered by 23% in the NAM group compared with placebo group, with effects in both BCC and SCC cancers. The ONTRAC study publication generated considerable scientific interest (19) (20).

Recently, the ONTRAC associates have focused on the use of NAM for the prevention of MEL (21). The 2015 ONTRAC study was not designed to report on the effect of oral NAM in prevention of MEL. However, during the ONTRAC 12 months study period, a total of six in situ and four invasive melanomas developed in the oral NAM and placebo groups. A significant increase in CD4⁺ helper T cells, and cytotoxic CD8⁺ T cells in the tumour microenvironment was reported in melanomas arising in the oral NAM group compared with placebo (22). We have found evidence from consumer acceptability studies in preliminary studies on subjects (see below) that glycol-coenhancer silicone dispersion (GCSD) topical dermatological formulations of 6%-10% NAM are able to prevent and treat sun-induced lesions and skin cancers to restore a normal skin. A scientific analysis, based on the observations described above, of NAM oral in-vivo and in vitro human skin epidermal pharmacokinetic studies and modelling is now presented to support our finding of unexpected clinical effects of GCSD formulations of NAM in treating and preventing skin cancers.

### Pharmacokinetic-Pharmacodynamic (PK-PD) Analysis of the Potential for Topical Dermatological NAM to be Effective to Treat and Prevent Skin Cancers

As we have commented, the ONTRAC study publication on the effect of oral doses of NAM in the range 500-1,000 mg/day to significantly prevent both BCC and SCC cancers generated considerable scientific interest. Also, NAM at such doses is without significant systemic adverse effects. Even so, there is a preference of users to use targeted topical therapy.

One particular advantage of topical therapy is that steady-state free concentrations may be achieved and sustained over a 6-8 hour period at the epidermal target site. This is in contrast to oral dosing where the rapid systemic clearance of NAM is such that, optimally, frequent re-dosing 4-6 times throughout the day may be required. If topical therapy can achieve therapeutic free NAM concentration at the basal epidermal target site, and sustain these over times approximating the dose interval, there may be significant advantage over oral therapy.

Skin Permeation of NAM from Glycol-Coenhancer Silicone Dispersions (GCSD)-1

NAM is a small molecule with a relatively low melting point and so, although it has a low octanol water partition coefficient, it penetrates through the lipophilic stratum corneum barrier, the rate of which may be increased by use of skin penetration mono-enhancer (23, 24) or co-enhancer technologies (25, 26).

Table 1 shows examples of GCSD cream formulations of the present invention. Example formulations were blinded as Test 1, Test 2 and Test 3 (as labelled in Table 1) and sent for independent analysis of in vitro permeation across human skin.

The permeation of NAM was tested for four formulations, namely Test 1, Test 2, Test 3 (respectively 6%, 8% and 10% NAM) and an 8% NAM commercial control cream. The study was conducted using Franz diffusion cells in human skin under finite dose conditions (10 μL/cm²) at 32±1° C. up to 24 h. The human (female, Caucasian) abdominal skin from one donor following plastic surgery was obtained from a tissue bank with institutional approval (Research Ethics Committee reference 07/H1306/98). The human skin was prepared by heat separation and skin integrity was confirmed by evaluation of electrical resistance (TM-22 Digitron digital thermometer, RS Components, UK) before use. Samples were collected at 0, 2, 4, 6, 8, 10, 12 and 24 h by withdrawing 200 μL of receptor medium and replacing with an equal volume of fresh PBS solution. At the end of the permeation study, the mass balance study was conducted following a procedure validated previously. The amounts of NAM in all samples were quantified using HPLC.

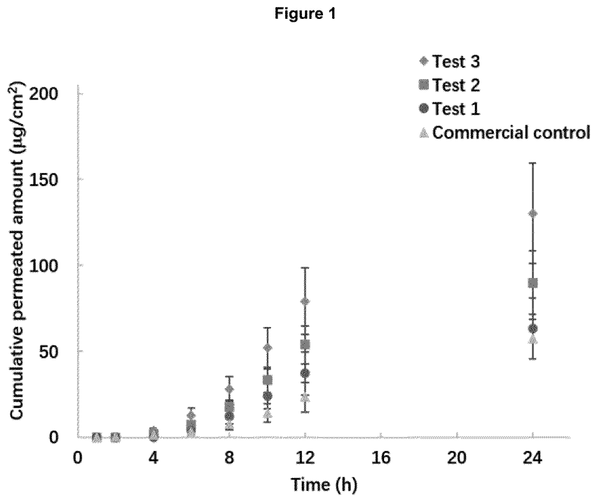

FIG. 1 shows permeation profiles of NAM 0-24 hours from tested formulations in human skin. Each data point represents the mean±SD, n=6. The results show the cumulative receptor concentrations of NAM from the four formulations over 24 hours.

There is a dose-response of cumulative permeation of NAM with time from Test 1 through to Test 3. This is driven (see Table 1) by increasing the concentration of propylene glycol, (the partition coefficient enhancer) from 30% to 35% to 40%, respectively. NAM is increased from 6% to 8% to 10% respectively, to maintain its degree of saturation in solution. The diffusion coefficient enhancer is a saturated solution in all three Test formulations. Addressing these three principles is essential for optimisation of the co-enhancer technology design.

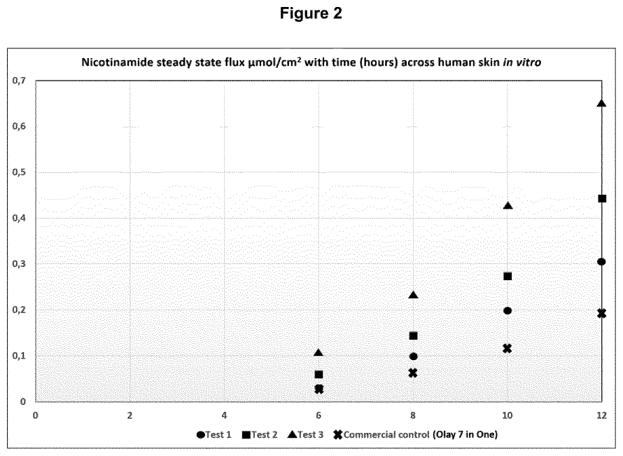

FIG. 2 shows the steady state permeation profiles of NAM 6-12 hours from tested formulations in human skin. Each data point represents the mean±SD, n=6. NOTE that mass units permeated per cm² are in μmol/cm² derived from FIG. 1 by dividing mass in ug by 122.1 the molecular weight of NAM. The results show the 6-12 hour steady state flux expressed as μmol/cm² derived from FIG. 1 by dividing mass in ug by the molecular weight of NAM.

From FIG. 2 the steady state flux over 6-12 hour may be calculated to be (Tests 1-3, commercial control respectively) 0.046, 0.064, 0.091 and 0.027 μmol/cm²/hour.

Estimation of Free NAM Concentrations in the Basal Epidermis from Steady State Flux Values

For this estimation it was assumed that the target site for BCC, SCC and MEL is the basal epidermis. The C star (C*) concept, relating the free drug concentration at the skin target site and the in vivo effectiveness of a topical formulation, was first developed using the antiviral acyclovir (27, 28) and was very successful in predicting in-vivo efficacy in animal models and also correlates well with clinical outcomes.

C* was calculated from equation 1. P_{D} was calculated from equations 2 and 3 (29) as recently modified by Pensado et al. (30).

C*=J/P_{D} (1)

P_{D}=D_{D}/h_{D} (2)

Dd=−4.15−(0.655×log MW) (3)

Where J is in vitro skin flux, P_{D} is the dermal permeability coefficient, D_{D} is the dermal diffusion coefficient and h_{D} is the thickness of the unperfused upper dermis. The permeability coefficient of NAM in the dermis was estimated using equation (2) where DOD, the dermal diffusion coefficient for free NAM was estimated from equation (4).

Log D_{DNAM}=−4.15−(0.655×log MW_{NAM}) (4)

Inputting the molecular weight of NAM of 122.12 into equation (4) gives a value for D_{DNAM} of 3.04×10⁻⁶ cm²s⁻¹. A value of 100 μm (0.01 cm) was used for h_{D}, the thickness of the unperfused upper dermis (30) and P_{D} calculated to be approximately 1.08 cm h⁻¹. For clarity of communication 3.04×10⁻⁶×60×60/0.01=1.08 cm h⁻¹.

As previously described, from FIG. 2, the steady state flux was calculated to be (Tests 1-3, commercial control respectively) 0.046, 0.064, 0.091 and 0.027 μmol/cm²/hour.

Thus, from equation (1), C*, the predicted free NAM concentrations at the target basal epidermal site are given by J, steady state flux in pmoles/cm²/hour, divided by P_{D}, thus are 0.043, 0.059, 0.084 and 0.025 μmol/cm³ for Test 1, Test 2 and Test 2 creams and the commercial control. Conversion from μmol/cm³ to μmol L⁻¹ gives 43, 59, 84 and 25 μmol L⁻¹. These concentrations are considerably higher than the concentrations of NAM, as low as 1-10 μmole L⁻¹, that very significantly increases the biosynthesis of ceramides and other stratum corneum lipids to increase skin barrier function in cosmetic applications.

Free NAM Concentrations Associated with Efficacy Against Skin Cancers

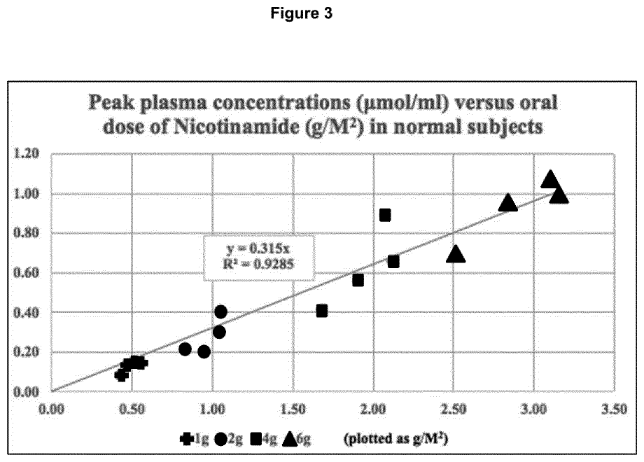

FIG. 3 shows a plot of peak plasma concentration, μmol/ml, of NAM plotted versus oral dose in g/M². FIG. 3 provides a plot of peak plasma concentrations, μmol/ml (cm³), following oral doses of NAM in g/M² administered to healthy volunteers by Stratford et al. (31).

Mean peak plasma concentrations from the 1 g (˜0.5 g/M²) dose were 0.127 μmol/ml (127 μmol L⁻¹); range 81-158 μmol L⁻¹). In the studies on oral NAM in the prevention of AK (17) and the ONTRAC study on BCC and SCC (18) a maximum oral dose of 500 mg of NAM twice a day was used which, assuming linearity, would be predicted to achieve peak plasma concentrations of approximately 63.5 μmol L⁻¹ ug/cm³; range ˜40-80 μmol L⁻¹. At steady state, and because NAM is poorly protein bound, similar concentrations of NAM are also predicted to be achieved in basal epidermal tissue and may provide a numerical target concentration to be achieved following topical administration.

As described, the Test 1, Test 2, Test 3 GCSD creams and the commercial control cream are predicted to achieve basal steady state epidermal concentrations of 43, 59, 84 and 25 μmol L⁻¹ respectively, which are in the range associated with the efficacy found in the ONTRAC study.

Therefore, the test creams having formulations of the present invention, following topical application to skin, achieve a peak plasma concentration of nicotinamide of above 40 μmol L⁻¹. Generally, the test creams and the formulations of the present invention, following topical application to skin, achieve a peak plasma concentration of nicotinamide of about 40 to about 80 μmol L⁻¹. This effect is superior to any other commercially available topical formulations.

Skin Permeation of NAM from Glycol-Coenhancer Silicone Dispersions (GCSD)-2

Table 2 shows examples of GCSD cream formulations of the present invention. Example formulations were blinded as Test #2.2, Test #4.2 and Test #6.2 (as labelled in Table 2) and sent for independent analysis of in vitro permeation across human skin alongside four commercially available nicotinamide containing skin products Olay, Paula's choice, TDF and SolarCareB3.

The NAM content of each formulation was assessed and the results shown in Table 3.

TABLE 3
NAM content in each tested formulation (mean ± SD, n = 5)

| Formulation | Assay (%, w/w) |
|---|---|
| TDF 1 | 4.61 ± 0.07 |
| Paula's choice | 10.00 ± 0.07 |
| Olay | 7.06 ± 0.08 |
| SolarCareB3 | 4.95 ± 0.04 |
| #2.2 | 6.48 ± 0.11 |
| #4.2 | 8.60 ± 0.38 |
| #6.2 | 10.87 ± 0.10 |

The permeation of NAM was tested as described above.

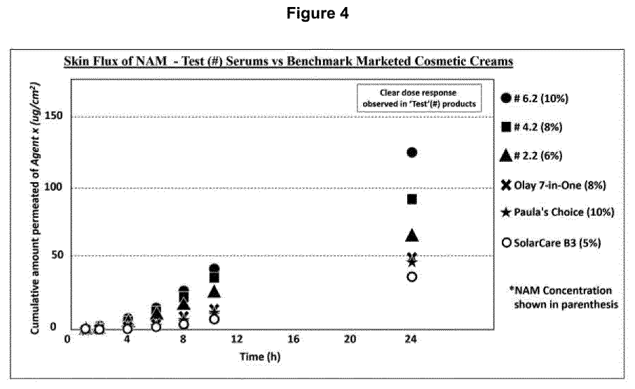

FIG. 4 shows the cumulative permeated amounts of NAM from formulations #2.2, 4.2, 6.2, TDF, Olay, Paula's choice and SolarCareB3. Each data point represents the mean±SD, n=6. The results show the cumulative receptor concentrations of NAM from the seven formulations over 24 hours.

The results from mass balance studies are summarized in Table 4. For all seven formulations, after 24 h of permeation, above 80% of applied NAM remained on the skin surface and was recovered by washing. No statistical difference was detected among these recovered percentage values by washing (p>0.05). The cumulative permeation of NAM from assessed formulations accounted for 4 to 17% of the applied dose. The permeated percentage values from formulations #6.2 and #4.2 was notably greater than the commercial products TDF, Olay, Paula's choice and SolarCareB3 (p<0.05). This shows that the formulations of the present invention offer significantly better permeation across the skin than that achieved by the commercial products also tested. There was no difference in NAM skin retention from formulations #6.2 (3.0±0.9%), 4.2 (2.4±0.4%) and 2.2 (2.8±0.4%, p>0.05). However, NAM retention from formulation #6.2, #4.2 and #2.2 was significantly higher compared to the results from Olay, Paula's choice and SolarCareB3 (p<0.05). The total recovery for all seven formulations were determined between 93.8 to 103.1%. These results fell in the mass balance criterion proposed by the Organisation for Economic Co-operation and Development (OECD), 90-110% [3].

TABLE 4
The results of mass balance studies following the permeation
studies of NIA from formulation #6.2, 4.2, 2.2, TDF,
Olay, Paula's choice, SolarCareB3 (n = 6, mean ± SD).

| | | Skin | | Total |
|---|---|---|---|---|
| | Washing | extraction | Permeation | recovery |
| Formulations | % | % | % | % |
| #6.2 | 82.3 ± 3.3 | 3.0 ± 0.9 | 16.9 ± 2.3 | 102.2 ± 3.9 |
| #4.2 | 84.1 ± 9.1 | 2.4 ± 0.4 | 16.7 ± 5.5 | 103.1 ± 8.5 |
| #2.2 | 89.8 ± 11.7 | 2.8 ± 0.4 | 11.2 ± 3.1 | 103.8 ± 12.8 |
| TDF | 83.8 ± 6.5 | 4.5 ± 0.4 | 12.6 ± 1.4 | 100.9 ± 7.5 |
| Olay | 84.6 ± 6.0 | 1.6 ± 0.3 | 7.7 ± 1.5 | 93.8 ± 6.4 |
| Paula's choice | 94.1 ± 5.8 | 1.1 ± 0.2 | 4.8 ± 1.2 | 100.0 ± 5.0 |
| SolarCareB3 | 89.0 ± 8.2 | 2.0 ± 0.6 | 8.8 ± 1.1 | 99.8 ± 7.0 |

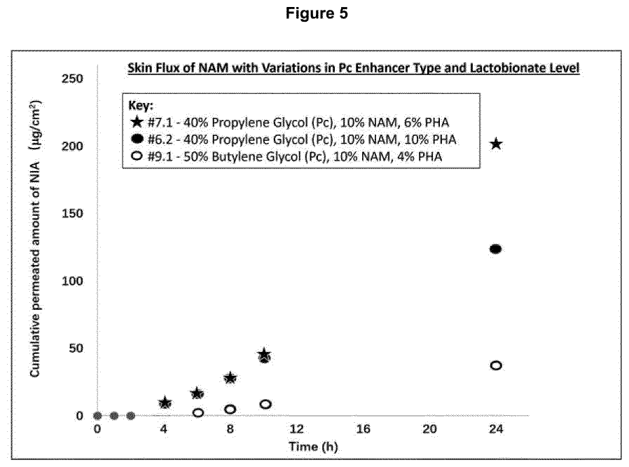

FIG. 5 shows the cumulative permeated amounts of NAM from formulations #7.1, #6.2 and #9.1. Each data point represents the mean±SD, n=6. The results show the cumulative receptor concentrations of NAM from the three formulations over 24 hours.

The results show that the permeated amount of NAM a greater for formulations #7.1, #6.2, which comprise propylene glycol that for formulation #9.1, which comprises butylene glycol. This result is despite more Pc enhancer being used in #9.1 (50%) than in #7.1 or #6.2 (both 40%). These data indicate that propylene glycol is significantly more favourable as the Pc enhancer vs butylene glycol.

### In Vitro Analysis for Risk of Skin Irritation

The formulations of Table 2 were also independently assessed in vitro for risk of causing skin irritation. The method used a validated test system EpiDerm™ reconstructed tissue model which is based on the effective time at which a material causes a 50% reduction in tissue viability (ET₅₀). The method involved topical application of 100 μl neat test item to the surface of the EpiDerm skin model. Exposure of the test items to skin model was for 1, 5, 18, 24 and 48 hours. For the negative control the skin model was left untreated for 5 h. For the positive control (100 μl of Triton X-100 1%) was applied for 3, 6 and 18 h.

ET₅₀ determination. After normalisation to the negative control (i.e. untreated negative control, percentage viability values were plotted on a graph with a semi-log x-axis and the ET₅₀ was determined.

Benchmark ET₅₀ values and groupings are shown in Table 5: As a general guideline, the following groupings were used in assigning expected in vivo irritancy responses based on the ET₅₀ results obtained using EPI-200.

TABLE 5

| ET₅₀ (h) | Expected in vivo irritancy |
|---|---|
| <0.5 | Strong/Severe, possible corrosive concentration |
| 0.5-4 | Moderate |
| 4-12 | Moderate to Mild |
| 12-24 | Very mild |
| 24 | Non-irritating |

Table 6 shows the ET₅₀ and corresponding classification for skin irritation from this experiment.

TABLE 6

| Test formulation | ET₅₀ (in hours) | Classification |
|---|---|---|
| Positive Control | 9.02 h | Moderate to mild irritant |
| #2.2 | 24.00 | Non-irritant |
| #4.2 | 18.00 | Very mild |
| #6.2 | 24.00 | Non-irritant |
| #7.1 | 18.00 | Very mild |
| #9.1 | 9.06 | Moderate to mild irritant |

In this test the positive control had an ET₅₀ value of 9.02 h which was correctly categorised as moderate to mild irritant. The formulations #2.2, #4.2 and #6.2 of the present invention are all classified as being non-irritant or very mild. The ET50 values obtained for test formulations #2.2, #4.2 and #6.2 are therefore between the values obtained by this methodology, and so comparable with, baby sunscreens. Therefore, it can be expected that the formulations of the present invention are non-irritating and can be used for extended periods, such as for years. Extend periods of use is recommended to prevent and treat conditions caused by UV exposure and skin cancers.

### Conclusions

Therefore it has been demonstrated that the present formulation achieves flux of nicotinamide into skin, following topical application, which matches the nicotinamide delivery levels previously achieved via oral delivery of nicotinamide in experiments showing prevention and/or treatment of skin cancers. Consequently, it has been demonstrated that the present formulation is expected to be effective in the prevention and/or treatment of skin cancers. It can be concluded that the present inventors have developed a topical formulation that is useful for preventing and/or treating a skin cancer selected from actinic keratosis, basal cell carcinoma, squamous cell carcinoma and melanoma. It can be concluded that the present inventors have developed a topical formulation for preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, and achieving complete or partial remission of basal cell carcinoma.

### Exemplification of Use—Preliminary Studies

The formulations containing 6%-10% NAM described herein may be structurally described as aqueous/glycol-coenhancer dispersions in a mixed silicone elastomer-silicone fluid continuous phase (GCSD). Glycol-coenhancer gels comprising, for example, propylene glycol as partition coefficient enhancer and a C₁₂₋₁₄ diffusion coefficient enhancer are known to be effective to increase skin penetration of an active but are associated with poor rub in and skin feel, tackiness and skin drying (32).

On application to the skin of glycol-coenhancer silicone dispersions (GCSD), the consumer experiences the soft and silky skin feel of the mixed silicone elastomer-silicone fluid continuous phase. Loss of volatile silicones occurs quickly to give the perception of absorption into the skin.

In order to get feedback on the aesthetics of this design, informal cosmetic studies on a number of subjects were undertaken.

### Feedback: Moisturisation

As a group, younger subjects' feedback was that GCSD products containing 6-10% Nicotinamide were exceptionally good moisturisers. This is to be expected from the design elements outline above.

### Feedback: Age Spots (Solar Lentigines), Sunspots and Liver Spots

These conditions occur after long-term exposure to UV light and so are associated with older subjects.

### Female Subject SJD

SJD reported sunspots on her hands due her time spent on many outdoor activities. She volunteered to undertake a study in which her left hand was treated and the right used as an untreated control. A cream formulation of the current invention containing 8% Nicotinamide was applied once a day up to 4 weeks to the left hand. After two weeks a noticeable evening of colour was reported with a significant reduction in the number of darker brown spots. The product was reported to be absorbed very quickly into the skin and left the skin feeling silky and soft.

### Female Subject JW

JW requested samples of a 6% Nicotinamide cream of the current invention for treatment of dry skin on hands and face. After approximately 4 weeks treatment she spontaneously mailed images of both hands. Her comment was that the sun spots had been very significantly reduced compared to pre-treatment.

### Feedback: Actinic Keratoses

Because of the informal and distant nature of these studies, and because of they are often mistaken for sunspots or age spots, it was not possible to get feedback on actinic keratoses (AK). In 2012, Surjana et al, (17) reported on two placebo-controlled studies in immune-competent volunteers actinic keratoses (AK) where statistically significant reductions in AK versus baseline (35% study 1; 29% study 2) were seen with the NAM groups (500 mg orally twice or once a day) compared with placebo. However, Moloney et al. (35) found no difference in AK scores after 6 months topical treatment with 1% Nicotinamide cream or placebo. Given the subsequent finding of Surjana et al, (17) with oral high dose oral NAM it seems likely that the low dose applied (and delivery efficiency) from the 1% NAM cream may have contributed to the lack of effect in AK.

### Feedback: Actinic Keratosis

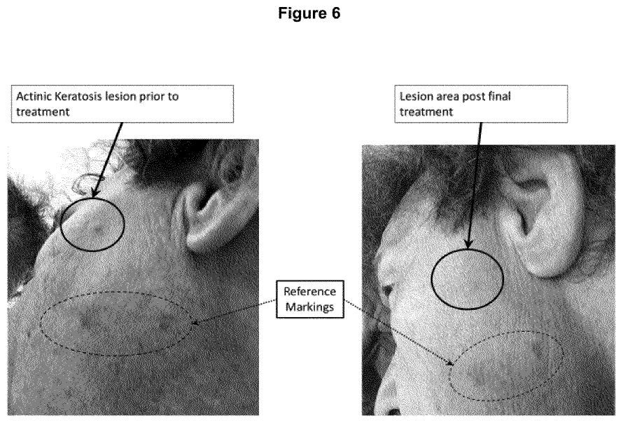

A 60-year-old white male volunteered to test a 10% nicotinamide containing cream of the present invention. The subject had been suffering for over a year with a solar keratosis lesion positioned on the left-hand side of his face in the vicinity of the upper side of the cheek bone as shown in FIG. 6 left hand panel. The lesion was characterised by an irregular edged area of reddish flaking skin around 0.5 cm in diameter with an elevated centre. The subject had often experienced a stinging or tingling sensation from the lesion. He had tried a range of emollients, moisturisers and medicated creams to alleviate the condition without any success.

The subject began by applying a liberal dose of the test cream to the lesion twice a day, but after two days reported that the lesion had started to sting and redden and he questioned if the product might be a bit too aggressive. He halted use of the cream for a day and noticing the area had calmed significantly he began re-applying the cream more sparingly only once per day for the rest of the week. Observing that no further irritation had occurred and that the lesion had improved, he resumed dosing twice a day. Significant improvement was observed over the next few weeks with the lesion gradually shrinking and the surface of the lesion smoothing to the level of the surrounding tissue as shown in FIG. 6 right hand panel.

After around two months the lesion had been reduced to little more than an area of slightly darker skin. The subject ceased use of the cream shortly after admitting that he assumed the lesion had been resolved and so he didn't need to bother further treatment. Nevertheless, on checking the area a few weeks later he was barely able to detect where the lesion had been. No deterioration of the area or evidence of reoccurrence was observed over the following twelve months.

### Feedback: Basal Cell Carcinoma

The same white male volunteer from the above Actinic Keratosis Example above, now 61 years of age, had begun to notice changes in what he considered a small innocuous ‘freckle’ on the upper right-hand side of his chest. Initially he noticed that it began to gradually increase in diameter to around one centimetre with an irregular broken edge. As time progressed the lesion developed raised areas and colour variations across the lesion. Crusting of areas also began to occur and some bleeding was noticed on towel drying after showering. The subject also began to notice evidence of overnight bleeding on bed linen.

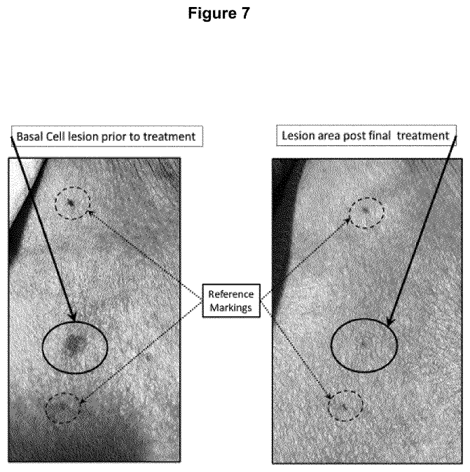

As the lesion progressed to develop a more sinister appearance the subject finally decided to visit his general medical practitioner, who promptly referred him to a consultant dermatologist specialising in skin cancer. On subsequent examination by the specialist a ‘basal cell carcinoma’ was diagnosed and the subject referred for surgical removal of the lesion. The basal cell carcinoma lesion is shown in FIG. 7 left hand panel.

Whilst waiting for surgery, and in the absence of any interim treatment, the subject decided to begin applying the 10% nicotinamide cream of the present invention, a sample of which he had retained from his previous testing on an actinic keratosis lesion. He admitted having no great confidence that the cream would do anything in this case, but felt he had nothing to lose in trying it as ultimately the lesion would be surgically removed.

He proceeded to apply the cream twice a day, noting in the early days that the lesion became rather flaky, but the level of redness did not increase at all. Over the next month he saw the lesion gradually shrink in size and become flattened and continuous with the surrounding normal skin. After around two months the original small ‘freckle’ was the most visible feature with a halo of ‘normal-looking’ skin but slightly lighter than the surrounding previously unaffected skin. The subject was pleased and surprised by the improvement achieved, and importantly he had avoided potentially disfiguring surgery and saved the health service some money in the process. The subject continued treatment for around three months before deciding that the lesion had been fully resolved. Twelve months on from treatment, aside from the obvious original ‘freckle’, the halo of the treated lesion remained barely detectable as shown in FIG. 7 right hand panel.

### Feedback: Melanoma

Fortunately, and as might be anticipated, no spontaneous reports of effect on MEL were obtained. However, as described herein it is expected that glycol-coenhancer silicone dispersion (GCSD) creams containing 6%-10% NAM may be effective to prevent and treat, to restore to normal skin, MEL lesions.

### Conclusions

Therefore it has been demonstrated that the present formulation advantageously delivers therapeutically effective levels of nicotinamide into the skin in the context of a formulation that is aesthetically acceptable and feels nice use. Therefore, whilst being effective the present formulation is also acceptable for long term use meaning that patient compliance is more readily achieved. Additionally, the present formulation has been demonstrated to show surprisingly good effects on skin cancers and other skin conditions caused by UV damage.

The present formulation represents the first consumer acceptable (and commercially viable) topical nicotinamide containing product that can achieve the target flux delivery levels required for the prevention of actinic keratosis and cancerous skin conditions.

The present formulation achieves this at commercially practical levels of 6-10% nicotinamide inclusion.

The present formulation achieves this whilst demonstrating high-end cosmetic aesthetics, rarely found in sun protection products, thus encouraging user compliance critical in achieving therapeutic potential.

The present formulation also incorporates synergistic benefits through inclusion of effective levels of Polyhydroxy acids which are known to deliver a range of benefits to skin, including moisturising and maintenance of the skin's natural barrier function. In short, SOL can encourage sustained daily use as part of an everyday skincare routine (so it represents much more than a product reserved for problem-solution sun protection).

The present formulation would be expected to provide protection against sun UV damage based on the known evidence, but totally unexpectedly, the present formulation has been found to be able to treat some cases of previous sun induced damage including brown-spot and actinic keratosis lesions and a diagnosed case of a basal cell carcinoma lesion. This is a surprising and very significant finding and extends the value of the present formulation into treatment as well as prevention.

### Summary of Studies

In preliminary cosmetic studies on a number of subjects described herein and undertaken to obtain feedback on the aesthetics of GCSD creams, we have in addition, and spontaneously, received feedback that these creams are exceptionally effective in treating sunspots and age spots and may be effective in treating skin cancers, particularly BCC. Why is this, as NAM has been applied topically, mainly in cosmetic use but also in medicinal use, for almost 50 years?

Concentration of NAM used in cosmetics have increased from 1-2% to up to 10% over the last decades, yet we know of no reports of efficacy of topical NAM to treat, to reverse, BCC, SCC or MEL skin cancers. This focus on increase in concentration of the active to increase skin permeation has been a major issue in topical formulation design. Unfortunately, the normal form of the Fickian permeation equation is:

J∝Cv*Pc*Dc

Where J is the flux, skin input rate, Cv is the concentration in solution in the vehicle and Pc and Dc are partition and diffusion coefficient terms. However, as Cv in solution is increased, for example by addition of a solvent for NAM, Pc is proportionally decreased, so that there is no net increase in J. Increasing concentration of NAM from 1-10% will not, on its own, increase NAM permeation.

NAM permeation may be increased by addition of skin penetration mono-enhancers (23, 24). The commercial control used in our in vitro human skin penetration study, developed by a major expert Global Player in this market, contains 8% NAM and the mono-skin penetration enhancer Octyl salicylate. Steady state basal cell concentration are predicted to be ˜25 μmol L⁻¹. A recent publication on a commercial mono-enhanced 4% NAM and two test 3% NAM formulations (36) allows steady state basal cell concentration in the range ˜9.50 to 17 μmol L⁻¹ to be predicted.

Very recently, Zang Y et al. 2019 (25) have described “simple” co-enhancer formulations of 5% NAM, in a combination of partition and diffusion coefficient enhancers such as propylene glycol and oleic acid or linoleic acid which show very similar efficiency of permeation of NAM to the co-enhancer GCSD creams (predicted basal cell concentrations of 35 μmol L⁻¹ under the finite dose conditions studied). However, these “simple” solvent mixtures are not, yet, complete formulations and it is unlikely that these research prototypes will have been used in vivo on human skin, at least for extended periods of time, such as to confirm our finding of the potential for NAM co-enhancer formulations to be effective to treat skin cancers.

In 2020 Malesu et al., the ONTRAC associates, (22) published on the potential of oral NAM to be chemoprevention on MEL. In their discussion, and from extrapolating the best line of fit to the data of Stratford et al. (31) (see FIG. 3 herein) they conclude that oral administration of 500 mg of NAM would achieve peak plasma concentrations of ˜80 μmol L⁻¹. In a previous publication (37) this group reported (see FIG. 6 of publication (37)) that in vitro NAM showed a dose-dependent inhibition of PARP-1 with 60% inhibition at a concentration of 50 μmol L⁻¹. PARP-1 inhibitors, based on nicotinamide structure, are currently undergoing clinical trials as targeted treatment modalities in the treatment of breast, uterine, colorectal and ovarian cancer.

As we have described, NAM containing glycol-coenhancer silicone dispersions (GCSD) are predicted to be able to achieve and sustain steady state free NAM concentrations of up to ˜80 μmol L⁻¹, a concentration that has been shown to significantly inhibit PARP-1. Also, the ability of topical administration of glycol-coenhancer silicone dispersion (GCSD) creams containing 6%-10% NAM to sustain steady-state concentration over a period of 12 hours may be an important benefit of the topical route as oral NAM is rapidly cleared from plasma (16).

While we believe that the above pharmacokinetic-pharmacodynamic arguments to support the potential for use of NAM containing glycol-coenhancer silicone dispersions (GCSD) to be effective in the prevention and treatment of skin cancers are sound, there may also be an adherence factor that contributes to the unexpected efficacy seen.

As discussed previously research into the reasons for low rates of adherence identifies that slow and poor response to treatment, incidence of or fear of incidence of local and systemic drug adverse effects (especially the phobia of topical corticosteroids), treatment regimens inconsistent with lifestyle and, specific to topical products, the poor aesthetics and experience in use of topical dermatological products, are main causal factors. The table below (Table 7) shows a summary of various dimensions effecting adherence.

TABLE 7
Dimensions of Adherence

| Patient social, | HCP-HC | | |
|---|---|---|---|
| economic and | system- | Disease- | Formulation |
| psychological | related | related | design-related |
| factors | factors | factors | factors |
| Age, gender, marital | Communication, | Visual | Dissatisfaction with |
| status, employment, | disease | (facial | Efficacy, local and |
| drug and alcohol | education, | lesions, | systemic AEs, |
| abuse | motivation | disease | excessive time and |
| | | severity | effort, poor sensory |
| | | | and perceptive |
| | | | experience in use |

Especially relevant to this discussion are the formulation cosmeceutical and biopharmaceutical design factors, briefly 1) efficacy, 2) local and systemic adverse effects potential, 3) time and effort in use (all as described by WHO) and 4) particularly the consumer experience in use.

On application to the skin of glycol-coenhancer silicone dispersions (GCSD), the consumer experiences the soft and silky skin feel of the mixed silicone elastomer-silicone fluid continuous phase. Loss of volatile silicones occurs quickly to give the perception of absorption into the skin.

GCSD formulations, as shown in FIGS. 1 and 2 and 4 herein, are exceptional in delivery of NAM when compared to current commercial NAM formulations. However, as important as NAM delivery to the target site is the experience in use of NAM GCSD creams equivalent to that experienced with Premium cosmetics; without adherence, there can be no therapeutic effect.

### REFERENCES

### Numbered References

1. Forbat, E, A I-Niaimi, F, Ali, F R. Use of nicotinamide in dermatology. Clin Exp Dermatol. 2017; 42(2):137-144.
2. Escudero-Gongora, M M, Fernandez-Penas, P. Nicotinamide: New Indications in Dermatology. Actas Dermosifiliogr. 2016; 107(9):777-778.
3. Chen, A C, Damian, D L. Nicotinamide and the skin. Australas J Dermatol. 2014; 55(3):169-175.
4. Wohlrab, J, Kreft, D. Niacinamide—mechanisms of action and its topical use in dermatology. Skin Pharmacol Physiol. 2014; 27(6):311-315.
5. Rolfe, H M. A review of nicotinamide: treatment of skin diseases and potential side effects. J Cosmet Dermatol. 2014; 13(4):324-328.
6. Namazi, M R. Nicotinamide in dermatology: a capsule summary. Int J Dermatol. 2007; 46(12):1229-1231.
7. Tanno, O, Ota, Y, Kitamura, N, Katsube, T, Inoue, S. Nicotinamide increases biosynthesis of ceramides as well as other stratum corneum lipids to improve the epidermal permeability barrier. Br J Dermatol. 2000; 143(3):524-531.
8. Soma, Y, Kashima, M, Imaizumi, A, Takahama, H, Kawakami, T, Mizoguchi, M. Moisturizing effects of topical nicotinamide on atopic dry skin. Int J Dermatol. 2005; 44(3):197-202.
9. Draelos, Z D, Ertel, K, Berge, C. Niacinamide-containing facial moisturizer improves skin barrier and benefits subjects with rosacea. Cutis. 2005; 76(2):135-141.
10. Kawada, A, Konishi, N, Oiso, N, Kawara, S, Date, A. Evaluation of anti-wrinkle effects of a novel cosmetic containing niacinamide. J Dermatol. 2008; 35(10):637-642.
11. Goon, A T, Tan, S H, Khoo, L S, Tan, T. Tetracycline and nicotinamide for the treatment of bullous pemphigoid: our experience in Singapore. Singapore Med J. 2000; 41(7):327-330.
12. Siadat, A H, Iraji, F, Khodadadi, M, Jary, M K. Topical nicotinamide in combination with calcipotriol for the treatment of mild to moderate psoriasis: A double-blind, randomized, comparative study. Adv Biomed Res. 2013; 2:90.
13. Iraji, F, Banan, L. The efficacy of nicotinamide gel 4% as an adjuvant therapy in the treatment of cutaneous erosions of pemphigus vulgaris. Dermatol Ther. 2010; 23(3):308-311.
14. Niren, N M. Pharmacologic doses of nicotinamide in the treatment of inflammatory skin conditions: a review. Cutis. 2006; 77(1 Suppl):11-16.
15. Watson, M, Holman, D M, Maguire-Eisen, M. Ultraviolet Radiation Exposure and Its Impact on Skin Cancer Risk. Semin Oncol Nurs. 2016; 32(3):241-254.
16. Damian, D L. Nicotinamide for skin cancer chemoprevention. Australas J Dermatol. 2017; 58(3):174-180.
17. Surjana, D, Halliday, G M, Martin, A J, Moloney, F J, Damian, D L. Oral nicotinamide reduces actinic keratoses in phase II double-blinded randomized controlled trials. J Invest Dermatol. 2012; 132(5):1497-1500.
18. Chen, A C, Martin, A J, Choy, B, Fernandez-Penas, P, Dalziell, R A, McKenzie, C A, Scolyer, R A, Dhillon, H M, Vardy, J L, Kricker, A, St George, G, Chinniah, N, Halliday, G M, Damian, D L. A Phase 3 Randomized Trial of Nicotinamide for Skin-Cancer Chemoprevention. N Engl J Med. 2015; 373(17):1618-1626.
19. Bagcchi, S. Nicotinamide yields impressive results in skin cancer. Lancet Oncol. 2015; 16:e591.
20. Barton, M K. Nicotinamide found to reduce the rate of nonmelanoma skin cancers in high-risk patients. CA Cancer J Clin. 2016; 66(2):91-92.
21. Minocha, R, Damian, D L, Halliday, G M. Melanoma and nonmelanoma skin cancer chemoprevention: A role for nicotinamide. Photodermatol Photoimmunol Photomed. 2018; 34(1):5-12.
22. Malesu, R, Martin, A J, Lyons, J G, Scolyer, R A, Chen, A C, McKenzie, C A, Madore, J, Halliday, G M, Damian, D L. Nicotinamide for skin cancer chemoprevention: effects of nicotinamide on melanoma in vitro and in vivo. Photochem Photobiol Sci. 2020; 19(2):171-179.
23. Iliopoulos, F, Sil, B C, Monjur Al Hossain, A S M, Moore, D J, Lucas, R A, Lane, M E. Topical delivery of niacinamide: Influence of neat solvents. Int J Pharm. 2020; 579:119137.
24. Zhang, Y, Lane, M E, Hadgraft, J, Heinrich, M, Chen, T, Lian, G, Sinko, B. A comparison of the in vitro permeation of niacinamide in mammalian skin and in the Parallel Artificial Membrane Permeation Assay (PAMPA) model. Int J Pharm. 2019; 556:142-149.
25. Zhang, Y, Kung, C P, Sil, B C, Lane, M E, Hadgraft, J, Heinrich, M, Sinko, B. Topical Delivery of Niacinamide: Influence of Binary and Ternary Solvent Systems. Pharmaceutics. 2019; 11(12)
26. Mohammed, D, Matts, P J, Hadgraft, J, Lane, M E. In vitro-in vivo correlation in skin permeation. Pharm Res. 2014; 31(2):394-400.
27. Mehta, S C, Afouna, M I, Ghanem, A H, Higuchi, W I, Kern, E R. Relationship of skin target site free drug concentration (C*) to the in vivo efficacy: an extensive evaluation of the predictive value of the C* concept using acyclovir as a model drug. J Pharm Sci. 1997; 86(7):797-801.
28. Imanidis, G, Song, W Q, Lee, P H, Su, M H, Kern, E R, Higuchi, W I. Estimation of skin target site acyclovir concentrations following controlled (trans)dermal drug delivery in topical and systemic treatment of cutaneous HSV-1 infections in hairless mice. Pharm Res. 1994; 11(7):1035-1041.
29. Kretsos, K, Miller, M A, Zamora-Estrada, G, Kasting, G B. Partitioning, diffusivity and clearance of skin permeants in mammalian dermis. Int J Pharm. 2008; 346(1-2):64-79.
30. Pensado, A, Chiu, W S, Cordery, S F, Rantou, E, Bunge, A L, Delgado-Charro, M B, Guy, R H. Stratum Corneum Sampling to Assess Bioequivalence between Topical Acyclovir Products. Pharm Res. 2019; 36(12):180.
31. Stratford, M R, Rojas, A, Hall, D W, Dennis, M F, Dische, S, Joiner, M C, Hodgkiss, R J. Pharmacokinetics of nicotinamide and its effect on blood pressure, pulse and body temperature in normal human volunteers. Radiother Oncol. 1992; 25(1):37-42.
32. A F, KAAD. Topical Formulation tf i WO 2011/070318 A2. RECKITT BENCKISER HEALTHCARE INTERNATIONAL LIMITED. 2011; UK
33. Draelos, Z D, Matsubara, A, Smiles, K. The effect of 2% niacinamide on facial sebum production. J Cosmet Laser Ther. 2006; 8(2):96-101.
34. Tasid-Kostov, M, Lukid, M, Savid, S. A 10% Lactobionic acid-containing moisturizer reduces skin surface pH without irritation—An in vivo/in vitro study. J Cosmet Dermatol. 2019; 18(6):1705-1710.
35. Moloney, F, Vestergaard, M, Radojkovic, B, Damian, D. Randomized, double-blinded, placebo controlled study to assess the effect of topical 1% nicotinamide on actinic keratoses. Br J Dermatol. 2010; 162(5):1138-1139.
36. Haque, T, Lane, M E, Sil, B C, Crowther, J M, Moore, D J. In vitro permeation and disposition of niacinamide in silicone and porcine skin of skin barrier-mimetic formulations. Int J Pharm. 2017; 520(1-2):158-162.
37. Park, J, Halliday, G M, Surjana, D, Damian, D L. Nicotinamide prevents ultraviolet radiation-induced cellular energy loss. Photochem Photobiol. 2010; 86(4):942-948.

### Further References

Skin cancer rates have soared by 45% in ten years in both young and old patients ‘because of rise of package holidays in the 1970s and a more recent surge in cheap flights’, Vanessa Chalmers MAILONLINE 18 Jul. 2019, https://www.dailymail.co.uk/health/article-7257449/Skin-cancer-rates-soared-45-10-years-package-holidays-cheap-flights.html
Skin Cancer Facts & Statistics, 2020 The Skin Cancer Foundation, https://www.skincancer.org/skin-cancer-information/skin-cancer-facts/
The Good, the Bad, and the Ugly of Sunscreens M Berwick, Clinical pharmacology & Therapeutics, p 31-33 VOLUME 89 NUMBER 1, JANUARY 2011
A Phase 3 Randomized Trial of Nicotinamide for Skin-Cancer Chemoprevention. Chen A C, Martin A J, Choy B, Fernández-Peñas P, Daiziell R A, McKenzie C A, Scolyer R A, Dhillon H M, Vardy J L, Kricker A, St George G, Chinniah N, Halliday G M, Damian D L. N Engl J Med. 2015 Oct. 22; 373(17):1618-26
New and current preventive treatment options in actinic keratosis P. Arenberger and M. Arenbergerova in JEADV 2017, 31 (Suppl. 5), 13-17
Could vitamin B-3 help to prevent melanoma? Honor Whiteman, Medical News Today, 9 Aug. 2017 https://www.medicalnewstoday.com/articles/318876.php
Melanoma and nonmelanoma skin cancer chemoprevention: A role for nicotinamide? Rashi Minocha, Diona L. Damian and Gary M. Halliday, Photodermatol Photoimmunol Photomed. 2017; 1-8
Nicotinamide for skin cancer chemoprevention, Diona L Damian, Australasian Journal of Dermatology (2017) 58, 174-180
Nicotinamide for skin cancer chemoprevention: effects of nicotinamide on melanoma in vitro and in vivo, Rashi Malesu, Andrew J. Martin, J. Guy Lyons, Richard A. Scolyer, Andrew C. Chen, Catriona A. McKenzie, Jason Madore, Gary M. Halliday and Diona L. Damian, Photochem. Photobiol. Sci., 2020, 19, 171-179

1. A topical formulation comprising:
a dispersed phase comprising:
5 to 15% w/w (percentage weight/weight) nicotinamide;
10 to 60% w/w of a partition coefficient enhancer (Pc enhancer), having a structure of the general formula: CₙH2n+202 where n represents an integer from 3 to 6 inclusive; and
≤10% water
and a continuous phase,
wherein the formulation has a pH of above 4.0.

2. The formulation according to claim 1, wherein the Pc enhancer is selected from the group consisting of propylene glycol, butylene glycol, pentylene glycol, hexylene glycol, and 1,5 pentane diol and a combination thereof.

3. The formulation according to claim 2, wherein the Pc enhancer is propylene glycol.

4. The formulation according to claim 1, wherein the formulation further comprises 2 to 10% w/w of a polyhydroxy acid.

5. The formulation according to claim 4, wherein the polyhydroxy acid is selected from gluconolactone, galactose and lactobionic acid.

6. The formulation according to claim 1, wherein the formulation further comprises about 0.5 to about 10% w/w of a co-enhancer, selected from the group consisting of a C₁₂ to C₁₄ straight chain fatty acid and a C₁₄ straight chain primary alcohol.

7. The formulation according to claim 1, wherein the continuous phase comprises:
5 to 45% w/w of a first dimethicone macromer mixture including a dimethicone macromer and a hydrocarbyl methyl siloxane emollient selected from the group consisting of an alkyl methyl siloxane, an aryl methyl siloxane and an alkyl aryl methyl siloxane, and
5 to 45% w/w of a second dimethicone macromer mixture including a methyl siloxane compound and a cross linked dimethicone macromer.

8. The formulation according to claim 1, wherein the formulation does not comprise one or more of the following components: tranexamic acid, zinc salt, zinc salt of a conjugate base of polyhydroxy acid, vitamin D, derivatives of vitamin D, and metabolites of vitamin D.

9. The formulation according to claim 1, wherein the formulation comprises a sun-blocking agent, a sun-screening agent or combinations thereof.

10. A pharmaceutical composition comprising the formulation according to claim 1.

11.-15. (canceled)

16. A method of preventing and/or treating a skin cancer, comprising:
administering the pharmaceutical composition of claim 10.

17. The method of claim 16, wherein the skin cancer is actinic keratosis, melanoma, squamous cell carcinoma, or basal cell carcinoma.

18. The method of claim 17, wherein the method comprises preventing actinic keratosis, treating actinic keratosis, preventing melanoma, preventing recurrence of melanoma, treating melanoma, achieving complete or partial remission of melanoma, preventing squamous cell carcinoma, preventing basal cell carcinoma, treating squamous cell carcinoma, treating basal cell carcinoma, achieving complete or partial remission of squamous cell carcinoma, or achieving complete or partial remission of basal cell carcinoma.

19. The method of claim 16, wherein the pharmaceutical composition is administered to the skin once a day or twice a day.

20. A cosmetic agent comprising:
the topical formulation of claim 1.
